(19) United States
(12) Patent Application Publication
(10) Pub. No.: US 2024/0366654 A1
(43) Pub. Date: Nov. 7, 2024

(54) PREBIOTIC COMPOSITION AND METHOD OF USE TO IMPROVE GASTROINTESTINAL HEALTH IN PATIENTS WITH DYSBIOSIS AND LEAKY GUT

(71) Applicants: Purdue Research Foundation, West Lafayette, IN (US); Rush University Medical Center, Chicago, IL (US)

(72) Inventors: Bruce R. Hamaker, West Lafayette, IN (US); Thaisa Moro Cantu Jungles, Lafayette, IN (US); Ali Keshavarzian, Evanston, IL (US)

(73) Assignees: Purdue Research Foundation, West Lafayette, IN (US); Rush University Medical Center, Chicago, IL (US)

(21) Appl. No.: 18/579,679

(22) PCT Filed: Jul. 15, 2022
(86) PCT No.: PCT/US22/37251

§ 371 Date: Jan. 16, 2024

Related U.S. Application Data
(60) Provisional application No. 63/222,562, filed on Jul. 16, 2021.

(51) Int. Cl. A61K 31/733; A61K 31/702; A61K 31/718; A61K 36/81; A61K 36/88; A61K 36/899; A61K 36/8998; A61P 1/14

(52) U.S. Cl. CPC A61K 31/733; A61K 31/702; A61K 31/718; A61K 36/81; A61K 36/88; A61K 36/899; A61K 36/8998; A61P 1/14

(57) ABSTRACT

A composition comprising (i) a resistant starch, (ii) a resistant non-starch α-linked glucan, (iii) a cereal bran, which is optionally stabilized, and (iv) inulin, a fructo-oligosaccharide, or both; an ingestible formulation comprising the composition; and a method of improving gastrointestinal health in a human with a condition, disease, or disorder, which method comprises administering to the human the composition or the ingestible formulation.

### CROSS REFERENCE TO RELATED APPLICATIONS

This application claims priority to U.S. provisional patent application No. 63/222,562, which was filed Jul. 16, 2021, and which is hereby incorporated by reference in its entirety.

### TECHNICAL FIELD

This disclosure relates to prebiotics, the gut microbiome, intestinal barrier function, dysbiosis, leaky gut, motor function, and diseases and conditions, such as chronic diseases and conditions, with dysbiosis and/or leaky gut, including, for example, neurodegenerative diseases, such as Parkinson disease.

### BACKGROUND

Non-communicable disorders (NCDs) are the primary cause of mortality and morbidity in developed and developing countries (Habib et al., Diabetes & Metabolic Syndrome: Clinical Research & Reviews 4.1: 41-47 (2010)). The underlying mechanism of most, if not all, NCDs is inflammation (Seyedsadjadi et al., Antioxidants 10(1): 15 (2020)). Converging evidence for epidemiological, clinical and experimental studies strongly suggests that the primary source of this inflammatory state is intestinal microbiota (Malesza et al., Cells 10(11): 3164 (2021); Bander et al., Int' J Environmental Res & Public Health 17(20): 7618 (2020); and Wang et al., Frontiers in Microbiol 11: 1065 (2020)).

Humans harbor a complex and rich bacterial community in their intestine (so called microbiota) that is intimately involved in metabolism and biological/physiological processes (Fan et al., Nature Reviews Microbiol 19(1): 55-71 (2021)). Thus, it is not surprising that disruption of the intestinal microbiota community (called “dysbiosis”) results in disruption of normal metabolism and biology. One critical negative impact of dysbiotic microbiota is disruption of the integrity of the normal intestinal barrier, which partitions intestinal luminal contents (which are directly in contact with the environment, primarily through diet and swallowed air) from the host, leading to intestinal leakage of the luminal contents, including bacterial products (Kinashi et al., Frontiers in Immunology, 12: 673708 (2021); and Fasano, F1000 Research 9 (2020)). Indeed, multiple studies have demonstrated abnormal microbiota (dysbiosis) and disrupted intestinal barrier function (leaky gut) in multiple NCDs, including two common gastrointestinal disorders, namely inflammatory bowel disease (e.g., ulcerative colitis, Crohn disease, and pouchitis) and irritable bowel syndrome (Camilerri et al., Neurogastroenterol Motil. 19(7): 545-552 (2007) 10.1111/j.1365-2982.2007.00925.x; Büning et al., Inflamm Bowel Dis. 18(10): 1932-1939 (2012); and Teahon et al., Gut 33(3): 320-323 (1992) 10.1136/gut.33.3.320), liver diseases including alcoholic liver disease and non-alcoholic steatohepatitis (NASH)—now the two leading causes of liver failure in the USA (Farhadi et al., Liver Int'l 28(7): 1026-1033 (2008); and Szabo, Gastroenterology 148(1): 30-36 (2015)), metabolic disorders like obesity, metabolic syndrome, and diabetes (Fasano, The Amer J of Clin Nutr 105(1): 3-4 (2017); and Chakaroun et al., Nutrients, 12(4), 1082 (2020)), cardiovascular diseases (Lewis, et al., Amer J of Physio-Heart and Circ Phys 319(6): H1227-H1233 (2020); and Manolis et al., Curr Med Chem (2022)), several cancers (Sánchez-Alcoholado, et al., Int'l J of Molec Sci 21(18): 6782 (2020); Sheflin et al., Curr Oncol Reports 16(10): 1-9 (2014); Xuan et al., PloS One (9(1): e83744 (2014); Sobhani et al., PloS One 6(1): e16393 (2011); Tsay et al., Cancer Disc 11(2): 293-307 (2021); and Bindels et al., Oncotarget 9(26): 18224 (2018)), and neurodegenerative diseases.

Neurodegenerative diseases include Parkinson disease (PD) and Alzheimer disease (Konjevod et al., J of Pharma and Biomed Analysis, 194: 113681 (2021); and Roe, Neurochem Res 1:14 (2021)), ataxia, Huntington disease, motor neuron disease, multiple system atrophy, neuromuscular disorders, Parkinsonism, post-traumatic stress disorder (PTSD), progressive supranuclear palsy, and spasticity, among others. Signs and symptoms of neurodegenerative disorders can affect mobility and balance, movement, swallowing, bladder and bowel function, sleep, breathing, heart function, memory and cognitive abilities, mood, and speech, for example. The Alzheimer's Association reported in 2022 that as many as 6.2 million people in the United States may have Alzheimer disease, whereas the Parkinson's Foundation reports nearly a million Americans are living with PD.

The likelihood of developing a neurodegenerative disease increases with age. As life expectancy increases, more and more individuals are expected to be affected by neurodegenerative disease.

Scientists recognize that genetics and environment contribute to the risk of developing neurodegenerative disease. Environmental factors, such as exposure to pesticides, fungicides, and insecticides, may play a role as well as exposure to metals (such as arsenic, lead and manganese), chemicals such as polychlorinated biphenyls (PCBs) and other chemicals used in industry and present in consumer products, pollution, and biological factors (such as endotoxins produced by bacteria).

Diet and lifestyle also can be environmental factors. An abnormal, pro-inflammatory intestinal microbiome promotes intestinal barrier dysfunction and systemic and neural inflammation, which collectively may influence neurodegenerative disease.

Whether sporadic or mono-genetic in origin, environmental factors may be critical in triggering PD onset in a susceptible host or influencing disease progression. Many environmental factors that are known to be risk factors for PD (e.g., diet, sleep, exercise, and stress) also influence the gut microbiome (Lauretti et al., Mol Psychiatry 22: 280-286 (2017); Marras et al., Movement disorders: official journal of the Movement Disorder Society 34: 801-811 (2019); Nag et al., Neurodegener Dis 19: 51-59 (2019); and van de Wouw (2018)). In fact, a growing body of evidence has implicated the intestinal microbiota as the trigger for microglial activation/neuroinflammation in PD (Sampson, “The Impact of indigenous microbes on Parkinson's disease,” Neurobiol Dis (2019); and Abdel-Haq et al., J Exp Med 216: 41-59 (2019)).

The intestinal microbiota influences brain development and function through a bidirectional communication known as the “gut-brain axis.” Recent studies document a disrupted intestinal microbiota community in multiple neurological diseases, such as PD (Bullich et al., Mov Disord Clin Pract 6: 639-651 (2019); and Park et al., “Regulation of common neurological disorders by gut microbial metabolites,” Exp Mol Med (2021)). Divergence of commensal bacteria composition from the microbial communities found in healthy individuals (termed “dysbiosis”) is associated with PD in both early and late stages (Kashavarzian et al., Prog Brain Res 252: 357-450 (2020); and Lubomski et al., “Parkinson's disease and the gastrointestinal microbiome,” J Neurol (2019)). Although there is no unique microbiota signature for PD, multiple studies have found that PD patients have a “pro-inflammatory” dysbiotic microbiota, characterized by decreased relative abundance of putative anti-inflammatory bacteria, including short chain fatty acid (SCFA)-producing bacteria and increased relative abundance of pro-inflammatory lipopolysaccharide (LPS)-producing bacteria (Keshavarzian et al., Movement disorders: official journal of the Movement Disorder Society 30: 1351-1360 (2015); Scheperjans et al., Movement disorders: official journal of the Movement Disorder Society 30: 350-358 (2015); Li et al., Front Mol Neurosci 12: 171 (2019); Aho et al., EBioMedicine 44: 691-707 (2019); Hill-Burns et al., Movement disorders: official journal of the Movement Disorder Society 32: 739-749 (2017); Huang et al., Front Cell Infect Microbiol 11: 615075 (2021); and Sun et al., Ageing Res Rev 45: 53-61 (2018)). These changes in the microbiota can augment systemic and neuroinflammation through several mechanisms including disruption of intestinal barrier integrity. SCFAs are critical in maintaining intestinal barrier integrity insomuch as barrier disruption (i.e., intestinal hyper-permeability) occurs when SCFA levels are low in the colon (Martin-Gallausiaux et al., Proc Nutr Soc 80: 37-49 (2021); Barbara et al., Front Nutr 8: 718356 (2021); Liu et al., Pharmacol Res 165: 105420 (2021); and Ma et al., Animal Nutr (2021)).

Intestinal barrier disruption permits the entry of pro-inflammatory bacterial components like LPS into the systemic circulation. Studies demonstrate that LPS activates microglia and promotes neurodegeneration (Brown, J Neuroinflammation 16: 180 (2019); and Lively et al., Front Cell Neurosci 12: 215 (2018)). Thus, the low SCFA levels in PD may promote intestinal leakiness leading to neuro-inflammation.

Studies suggest that microbiota-directed interventions (prebiotics (mainly indigestible carbohydrates to promote growth of “beneficial” bacteria), probiotics (“beneficial” live bacteria), and fecal microbiota transplant (FMT)) might beneficially impact symptoms and/or PD pathogenesis (Van Laar et al., J Parkinson's Dis 9: S371-S379 (2019); and Walsh et al., FEBS Letters 588(22): 4120-4130 (2014)). A microbiota-directed intervention that increases SCFA could fortify intestinal barrier integrity and dampen neuroinflammation in PD patients, thereby modifying PD disease course. One such microbiota-directed intervention is prebiotic fibers (Cantu-Jungles et al., Front Neurol 10: 663 (2019)). Dietary fibers are not hydrolyzed by mammalian enzymes and arrive intact in the colon, where they are fermented by the bacteria in the colon. Each bacterial group has a preference regarding physical and chemical characteristics of fibers, and this information has been leveraged to develop a mixture of prebiotic fibers that promotes the growth of distinct groups of bacteria associated with health benefits, including the production of SCFA (Cantu-Jungles et al. (2019), supra; Rose et al., Nutr Rev 65: 51-62 (2007); Bishehsari et al., “Dietary Fiber Treatment Corrects the Composition of Gut Microbiota, Promotes SCFA Production, and Suppresses Colon Carcinogenesis,” Genes (Basel) 9 (2018); Kaur et al., Mol Nutr Food Res 63: e1801012 (2019); and Hamaker et al., J Mol Biol 426: 3838-3850 (2014)). Prebiotic fibers are generally regarded as safe (GRAS) and have been used for centuries to treat chronic illnesses and are capable of modifying microbiota communities (Cantu-Jungles (2019), supra; Hutkins et al., Curr Opin Biotechnol 37: 1-7 (2016)). However, there do not appear to be any reports of using a prebiotic mixture designed to augment SCFA production in patients with PD (Cantu-Jungles (2019), supra).

The success of any strategy is based on whether the intervention can directly address the abnormality that requires fixing. Thus, a careful understanding of what aspects of dysbiosis could be the primary driver of systemic inflammation is critical to design a successful intervention. Several studies have demonstrated that intestinal leaking of luminal contents, including pro-inflammatory bacterial and dietary metabolites, appears to be a key driver of systemic inflammation in NCDs.

Herein is described a novel approach to modulate the gut microbiota and increase SCFA production resulting in improved intestinal barrier function and reduced local and systemic inflammation. The approach is exemplified using Parkinson disease (the second most common neurodegenerative disease in the USA, which is characterized by a low abundance of SCFA-producing bacteria and associated with dysbiosis and leaky gut) to demonstrate how the approach can be used to prevent/treat multiple NCDs in which dysbiosis, characterized by a low abundance of SCFA-producing bacteria, and/or leaky gut is/are present.

In view of the above, it is an object of the present disclosure to provide a prebiotic fiber composition for administration to at risk for, or having, an NCD characterized by dysbiosis and/or leaky gut. This and other objects and advantages, as well as inventive features, will be apparent from the detailed description provided herein.

### SUMMARY

Provided is a composition comprising (i) a resistant starch, (ii) a resistant non-starch α-linked glucan, (iii) a cereal bran, which is optionally stabilized, and (iv) inulin, a fructo-oligosaccharide, or both. The resistant starch can be type 1, type 2, type 3, type 4, type 5, or any combination thereof. The resistant starch can be type 2. The type 2 resistant starch can be raw potato starch. The resistant non-starch α-linked glucan can be resistant dextrin/maltodextrin. The cereal bran can be rice bran, wheat bran, corn bran, oat bran, barley bran, sorghum bran, millet bran, rye bran, triticale bran, or any combination thereof. The cereal bran can be rice bran, which is optionally stabilized. The inulin can be agave branched inulin. In an embodiment, the composition can comprise raw potato starch, resistant dextrin/maltodextrin, rice bran, which is optionally stabilized, and agave branched inulin. Each of (i)-(iv) can be present in an amount in the range of about 5% to about 70% of the total amount by weight. IN an embodiment, the composition comprises about 30% type 2 resistant starch, about 30% resistant maltodextrin, about 30% rice bran, which is optionally stabilized, and about 10% agave branched inulin.

Further provided is an ingestible formulation comprising an above-described composition. The ingestible formulation can comprise from about 2 grams to about 20 grams of the composition. The ingestible formulation can be a supplement, a powder sachet, a powder for a shake, a liquid shake, a prebiotic shot, a snack, or a meal replacement.

Still further provided is a method of improving gastrointestinal health in a human with a condition, disease, or disorder. The method comprises administering to the human an above-described composition or an ingestible formulation comprising same. The ingestible formulation can comprise from about 2 grams to about 20 grams of the composition. The ingestible formulation can be a supplement, a powder sachet, a powder for a shake, a liquid shake, a prebiotic shot, a snack, or a meal replacement. The ingestible formulation can be administered at least once daily. The ingestible formulation can be administered twice daily. The human can have an inflammatory bowel disease, irritable bowel syndrome, liver disease, a metabolic disorder, a cardiovascular disease, a cancer, a neurodegenerative disease, an infection, a condition induced by exposure to chemotherapy or radiation, or an allergy. The inflammatory bowel disease can be ulcerative colitis, Crohn disease, or pouchitis. The liver disease can be alcoholic liver disease or non-alcoholic steatohepatitis (NASH). The metabolic disorder can be obesity, metabolic syndrome, or diabetes. The neurodegenerative disease can be Parkinson disease, Alzheimer disease, ataxia, Huntington disease, motor neuron disease, multiple system atrophy, a neuromuscular disorder, Parkinsonism, post-traumatic stress disorder (PTSD), progressive supranuclear palsy, or spasticity. The infection can be a viral infection. The viral infection can be human immunodeficiency virus infection. The condition induced by exposure to chemotherapy or radiation can be enteritis. Each of (i)-(iv) in the composition can be present in an amount in the range of about 5% to about 70% of the total amount by weight. The composition can comprise about 30% type 2 resistant starch, about 30% resistant maltodextrin, about 30% rice bran, which is optionally stabilized, and about 10% agave branched inulin.

### BRIEF DESCRIPTION OF THE FIGURES

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1A shows hierarchical clustering of the 25 most abundant genera (heatmap represents log2 relative abundance) after 24 hours of in vitro fecal fermentation. Hierarchical clustering was performed using Euclidean distances and the Ward algorithm, and clusters of taxa were associated with fiber types.

FIG. 1B is a bag graph of fiber type vs. mM/50 mg carbohydrate, which shows the total short-chain fatty acids (SCFAs) produced during 24 hours of in vitro fecal fermentation. Bars denoted by different letters indicate significant differences between treatment means (p<0.05).

FIG. 1C is a bar graph of fiber type vs. mM/50 mg carbohydrate, which shows acetate produced during 24 hours of in vitro fecal fermentation.

FIG. 1D is a bar graph of fiber type vs. mM/50 mg carbohydrate, which shows butyrate produced during 24 hours of in vitro fecal fermentation.

FIG. 1E is a bar graph of fiber type vs. mM/50 mg carbohydrate, which shows propionate produced during 24 hours of in vitro fecal fermentation.

FIG. 1F is a bar graph of fiber type vs. SCFA proportion (%), which shows the proportion of butyrate, propionate, and acetate produced during 24 hours of in vitro fecal fermentation.

FIG. 2A is a table showing the diversity indices of Shannon Index, Simpson's Index, Species Richness, and Pielou's Evenness as measured at the taxonomic level of species. Mean index score and standard deviation (SD) are displayed.

FIG. 2B shows centroids representing the mean values of the baseline and prebiotic groups. Non-metric MDS plots were built on Bray-Curtis dissimilarity metrics.

FIG. 2C shows the mean relative abundance of fecal microbial communities at baseline and after prebiotic intervention (n=20). The mean relative abundance of taxa with greater than 1% average relative abundance are shown. Differentially abundant taxa are bolded (Wilcoxon signed rank pair-test: p<0.05).

FIG. 2D is a bar graph of feces (baseline (BL) and after prebiotic intervention (prebiotic) vs. Proteobacterial proinflammatory-producing bacteria mean abundance (%). Wilcoxon signed rank pair test, *p<0.05; **p<0.01; ***p<0.001.

FIG. 2E is a bar graph of feces (BL and prebiotic) vs. Escherichia coli proinflammatory-producing bacteria mean abundance (%). Wilcoxon signed rank pair test, *p<0.05; **p<0.01; ***p<0.001.

FIG. 2F is a bar graph of feces (BL and prebiotic) vs. Faecalibacterium praunsnitzii SCFA-producing bacteria mean abundance (%). Wilcoxon signed rank pair test, *p<0.05; **p<0.01; ***p<0.001.

FIG. 2G is a bar graph of feces (BL and prebiotic) vs. Bifidobacterium adolescentis SCFA-producing bacteria mean abundance (%). Wilcoxon signed rank pair test, *p<0.05; **p<0.01; ***p<0.001.

FIG. 2H is a bar graph of feces (BL and prebiotic) vs. Fusicatenibacter saccharivorans SCFA-producing bacterial mean abundance (%) (Log 10). Wilcoxon signed rank pair test, *p<0.05; **p<0.01; ***p<0.001.

FIG. 2I is a bar graph of feces (BL and prebiotic) vs. Ruminococcus bicirculans SCFA-producing bacteria mean abundance (%) (Log 10). Wilcoxon signed rank pair test, *p<0.05; **p<0.01; ***p<0.001.

FIG. 2J is a bar graph of feces (BL and prebiotic) vs. Parabacteroides merdae SCFA-producing bacteria mean abundance (%) (Log 10). Wilcoxon signed rank pair test, *p<0.05; **p<0.01; ***p<0.001.

FIG. 2K is a bar graph of feces (BL and prebiotic) vs. plasma total SCFA (μg/mL). Wilcoxon signed rank pair test, *p<0.05; **p<0.01; ***p<0.001.

FIG. 3A is a bar graph of plasma (BL and prebiotic) vs. zonulin (ng/ml), which is an intestinal barrier marker of leaky gut. Wilcoxon signed rank pair test, *p<0.05; **p<0.01.

FIG. 3B is a bar graph of plasma (BL and prebiotic) vs. calprotectin (μg/g), which is a marker of intestinal inflammation. Wilcoxon signed rank pair test, *p<0.05; **p<0.01.

FIG. 3C is a bar graph of plasma (BL and prebiotic) vs. neurofilament light chain (NfL; μg/g), which is a peripheral marker of neuro-axonal injury (i.e., neurodegeneration). Wilcoxon signed rank pair test, *p<0.05; ** p<0.01.

### DETAILED DESCRIPTION

For the purposes of promoting an understanding of the principles of the present disclosure, reference will now be made to the embodiments illustrated in the figures, and specific language will be used to describe the same. No limitation of scope is intended by the description of these embodiments. On the contrary, this disclosure is intended to cover alternatives, modifications, and equivalents as may be included within the spirit and scope of this application as defined by the appended claims.

The present disclosure is predicated, at least in part, on the discovery that a multi-targeted prebiotic fiber mixture can safely modify the intestinal microbiome, improve the intestinal barrier, blunt neuroinflammation, and influence disease severity in patients with Parkinson's disease (PD), including those early in the disease before initiating medication (i.e., de novo) and those with more advanced disease and being treated with levodopa.

Provided is a composition. The composition comprises (i) a resistant starch, (ii) a resistant non-starch α-linked glucan, (iii) a cereal bran, which is optionally stabilized, and (iv) inulin, a fructo-oligosaccharide, or both.

The resistant starch can be type 1, type 2, type 3, type 4, type 5, or any combination thereof. Example combinations include, but are not limited to, types 1 and 2, types 1 and 3, types 1 and 4, types 1 and 5, types 2 and 3, types 2 and 4, types 2 and 5, types 3 and 4, types 3 and 5, types 4 and 5, types 1-3, types 2-4, types 3-5, types 1-4, types 2-5, types 1-5, or type 2 and one or more types selected from types 1 and 3-5. Type 1 starch can be found in partially milled seeds and grains. Type 1 resistant starch can also be found in some dense, starch foods. Type 1 resistant starch can be associated with fibrous cell wells.

In various embodiments, the resistant starch is type 2. Resistant starch type 2 (RS2) is naturally resistant (i.e., resistant to digestion in the small intestine) because it is granular. The granules are mostly B-type (X-ray pattern) starches, which are compact and resistant to enzymatic degradation. RS2 occurs in foods where the starch is raw or where the granules do not gelatinize during cooking. RS2 is butyrogenic. Examples of RS2 include, but are not limited to, raw potato, green banana, some legumes, and high amylose starch, such as corn starch (e.g., Eurylon®, Novelose240®, Hylon® VII, and Hi-maize™). The type 2 resistant starch can be raw potato starch.

Type 3 resistant starch is the most resistant type of starch. It can be found in food that has been cooked and cooled, such as bread and boxed cereal.

Type 4 resistant starch is man-made. It is usually found in baked goods.

Type 5 resistant starch is a starch-lipid V-type complex, such as starch-fatty acids and starch-monoglycerides. An example is an amylose-lipid complex, which is resistant to enzyme hydrolysis. Other examples include starch-glycerol, starch-amino acids, starch-peptides, starch-proteins, starch-lipid protein, starch-polyphenols, starch-other polysaccharides, and the like.

Resistant non-starch α-linked glucans (or α-glucans) are polysaccharides of D-glucose monomers linked with glycosidic bonds of the α form. The resistant non-starch α-linked glucan can be resistant dextrin/maltodextrin. Resistant dextrin/maltodextrin is a non-viscous dietary fiber classified as resistant starch type V. It is made from corn starch by controlled conversion of digestible glucose constituents into constituents that are resistant to digestion in the small intestine. Resistant maltodextrin is propiogenic. The resistant dextrin/maltodextrin can be Nutriose™ (Roquette Co.), Promitor (Tate & Lyle), or Fibersol-2 (ADM), for example.

The cereal bran can be any suitable cereal brain. In various embodiments, the cereal bran is rice bran, wheat bran, corn bran, oat bran, barley bran, sorghum bran, millet bran, rye bran, triticale bran, or any combination thereof. The cereal bran can be optionally stabilized. In an embodiment, the cereal bran is rice bran, which is optionally stabilized. Stabilized rice bran is rice bran that has been treated. Most commonly the rice bran is heated above 120° C. under dry or moist conditions to denature the enzyme responsible for oxidation of the oil (about 20% of rice bran) in rice bran without destroying the nutritional value of the rice bran.

The inulin can be any suitable inulin. In an embodiment, the inulin is agave branched inulin, which is extracted from Agave tequilana, a species of blue agave native to Mexico. Agave inulin has short and long branched chain fructans. The branched chain fructans are used herein. All three short chain fatty acids (SCFAs), namely acetate, butyrate, and propionate, increase with administration of inulin and rice bran. The most total SCFAs, however, result when inulin is used in combination with resistant maltodextrin.

In an embodiment, the composition can comprise raw potato starch, resistant dextrin/maltodextrin, rice bran, which is optionally stabilized, and agave branched inulin.

Each of (i)-(iv) can be present in an amount in the range of about 5% to about 70% of the total amount by weight of the composition. For example, each of (i)-(iv) can be present in an amount in the range of 5% to about 70% or about 5% to 70% of the total amount by weight. Thus, each of (i)-(iv) can be present in an amount of 5%, 7.5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, or 70%, as well as any other whole or fractional percentage in the range of about 5% to about 70%, of the total amount by weight of the composition.

In an embodiment, the composition comprises about 30%, such as 30%, type 2 resistant starch, about 30%, such as 30%, resistant maltodextrin, about 30%, such as 30%, rice bran, which is optionally stabilized, and about 10%, such as 10%, agave branched inulin.

Further provided is an ingestible formulation comprising an above-described composition. The ingestible formulation can comprise from about 2 grams to about 20 grams (such as about 2 grams to 20 grams or 2 grams to about 20 grams, e.g., 2-19, 2-18, 2-17, 2-16, 2-15, 2-14, 2-13, 2-12, 2-11, 2-10, 2-9, 2-8, 2-7, 2-6, 2-5, 2-4, 2-3, 5-15, 5-10, 3-20, 4-20, 5-20, 6-20, 7-20, 8-20, 9-20, 10-20, 11-20, 12-20, 13-20, 14-20, 15-20, 16-20, 17-20, 18-20, or 19-20) of the composition. The ingestible formulation can be any suitable ingestible formulation as known in the art, examples of which include a supplement, a powder sachet, a powder for a shake, a liquid shake, a prebiotic shot, a snack, or a meal replacement.

The preparation of prebiotic fibers and compositions comprising same is within the ordinary skill in the art. Likewise, the preparation of ingestible formulations comprising a composition comprising prebiotic fibers is also within the ordinary skill in the art.

Still further provided is a method of improving gastrointestinal health in a human with a condition, disease, or disorder, any one of which can be chronic. “Improving gastrointestinal health” includes, but is not limited to, increasing beneficial bacteria in the gut microbiota (e.g., decreasing dysbiosis), increasing SCFA production, improving intestinal barrier function (e.g., improving leaky gut), and/or reducing local and/or systemic inflammation. The method comprises administering to the human an above-described composition or an ingestible formulation comprising same. The ingestible formulation can comprise from about 2 grams to about 20 grams (such as about 2 grams to 20 grams or 2 grams to about 20 grams, e.g., 2-19, 2-18, 2-17, 2-16, 2-15, 2-14, 2-13, 2-12, 2-11, 2-10, 2-9, 2-8, 2-7, 2-6, 2-5, 2-4, 2-3, 5-15, 5-10, 3-20, 4-20, 5-20, 6-20, 7-20, 8-20, 9-20, 10-20, 11-20, 12-20, 13-20, 14-20, 15-20, 16-20, 17-20, 18-20, or 19-20) of the composition. The ingestible formulation can be a supplement, a powder sachet, a powder for a shake, a liquid shake, a prebiotic shot, a snack, or a meal replacement. The ingestible formulation can be administered at least once daily. The ingestible formulation can be administered twice daily. The human can have any condition, disease, or disorder (any of which can be chronic) in which the improvement of gastrointestinal health has a beneficial effect. Examples of conditions, diseases and disorders include, but are not limited to, an inflammatory bowel disease, irritable bowel syndrome, liver disease, a metabolic disorder, a cardiovascular disease, a cancer, a neurodegenerative disease, an infection, a condition induced by exposure to chemotherapy or radiation, or an allergy. The inflammatory bowel disease can be ulcerative colitis, Crohn disease, or pouchitis. The liver disease can be alcoholic liver disease or non-alcoholic steatohepatitis (NASH). The metabolic disorder can be obesity, metabolic syndrome, or diabetes. The neurodegenerative disease can be Parkinson disease, Alzheimer disease, ataxia, Huntington disease, motor neuron disease, multiple system atrophy, a neuromuscular disorder, Parkinsonism, post-traumatic stress disorder (PTSD), progressive supranuclear palsy, or spasticity. The infection can be a viral infection. The viral infection can be human immunodeficiency virus infection. The condition induced by exposure to chemotherapy or radiation can be enteritis. Each of (i)-(iv) in the composition can be present in an amount in the range of about 5% to about 70% (such 5% to about 70% or about 5% to 70%) of the total amount by weight. Thus, each of (i)-(iv) can be present in an amount of 5%, 7.5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, or 70%, as well as any other whole or fractional percentage in the range of about 5% to about 70%, of the total amount by weight of the composition. The composition can comprise about 30%, such as 30%, type 2 resistant starch, about 30%, such as 30%, resistant maltodextrin, about 30%, such as 30%, rice bran, which is optionally stabilized, and about 10%, such as 10%, agave branched inulin.

Administration of the composition or an ingestible formulation comprising the composition to a human with Parkinson's disease can improve gastrointestinal health in a subset of levodopa-treated patients, improve motor dysfunction, decrease inflammatory bacteria in the intestinal microbiome (e.g., Protebacteria and E. coli), increase SCFA-producing bacteria in the intestinal microbiome (e.g., Faecalibacterium prausnitizii, Bifidobacterium adolescentis, Ruminococcus bicirculans, Fusicatenibacter saccharivorans, and Parabacteroides merdae), increase total SCFA metabolites, increase total SCFA in plasma, increase acetate, propionate, and butyrate, increase the ratio of [propionate+butyrate]:[total SCFA], improve intestinal barrier integrity in a patient being treated with levodopa (e.g., as evidenced by a decrease in plasma zonulin), reduce the level of neurofilament light chain (NfL; peripheral marker of neurodegeneration) in a de novo patient, and/or reduce intestinal inflammation (e.g., as evidenced by a decrease in fecal calprotectin). Total SCFA metabolites, intestinal barrier integrity, NfL level, and intestinal inflammation can be assessed by blood assays. Administration of the composition or a snack or meal replacement comprising same to a human with Parkinson's disease can decrease total Unified Parkinson's Disease Rating Scale (UPDRS) score, and/or down-regulate acetyl COA fermentation to butanoate II.

### EXAMPLES

The following examples serve to illustrate the present disclosure. The examples are not intended to limit the scope of the claimed invention.

### Example 1

### In Vitro Studies: Dietary Fiber Impact on Microbiota Structure/Function

Rationale: Four prebiotic fibers (inulin, resistant starch type 2, resistant maltodextrin, and rice bran) were chosen to be tested in vitro to evaluate their potential to increase the abundance of groups of bacteria associated with health benefits and promote SCFA production. The fibers are slow fermenting, making them tolerable for in vivo use and because slow fermentation allows them to be delivered to the distal parts of the colon (So (2021)).

Procedure: A 24 h in vitro human fecal fermentation with each individual fiber was performed as previously described (Tuncil et al., J Funct Foods 32: 347-357 (2017); and Cantu-Jungles et al., Carbohydr Polym 183: 219-229 (2018)). Briefly, carbonate-phosphate buffer was prepared and sterilized by autoclaving (121° C., 20 min). The buffer was then cooled to room temperature, oxygen was removed by bubbling with carbon dioxide, and cysteine hydrochloride (0.25 g/liter of buffer) was added as a reducing agent. The buffer was then placed into the anaerobic chamber the day before experimentation to complete buffer reduction. On the day of experiment, freshly collected fecal samples from three healthy human donors (10 g/each) were homogenized with carbonate-phosphate buffer (1:3 [wt/vol]), followed by filtration through four layers of cheesecloth. Then, 1 ml of this pooled fecal inoculum was added to Balch-type tubes containing 50 mg of dietary fiber substrate and 4ml of the carbonate-phosphate buffer. Tubes were closed with butyl rubber stoppers, sealed with aluminum seals, and incubated for 24 h (37° C. on a shaker inside an incubator (150 rpm)). All sample manipulation was conducted in an anaerobic atmosphere (85% N₂, 5% CO₂, and 10% H₂).

Microbiota Analysis: Fermented fecal inoculum samples were centrifuged (20,784(rcf)/(g), 15 min), and supernatant discarded. Automated DNA extraction of the precipitate was performed using the QIAcube Connect instrument (Qiagen, Germantown, MD) with the QIAamp PowerFecal Pro DNA kit (Qiagen, Germantown, MD) per manufacturer instructions. The V4 region of 16S rRNA gene was amplified and then sequenced using the Illumina MiniSeq platform (Illumina, Inc., San Diego, CA), as previously described (Cantu-Jungles et al., mBio 12: e0102821 (2021)). Library preparation and 16S rRNA gene sequencing were performed at the DNA Services Facility at the University of Illinois at Chicago (Chicago, IL).

SCFA Analysis: Fermented fecal inoculum samples were prepared as previously described (Cantu-Jungles (2018), supra), and analyzed at Purdue University using a gas chromatograph (GC-FID 7890 A; Agilent Technologies Inc.) on a fused silica capillary column (Nukon Supelco no. 40369-03A; Bellefonte, PA) under the following conditions: injector temperature of 230° C., initial oven temperature at 100° C., and temperature increase of 8° C./min to 200° C. with a hold for 3 min at final temperature. Helium was used as a carrier gas at 0.75 ml/min. Quantification was performed based on relative peak area using external standards of acetate (A38S), propionate (A258), and butyrate (AC108111000) and an internal standard of 4-methylvaleric acid (AAA1540506) from Fisher Scientific (Hampton, NH).

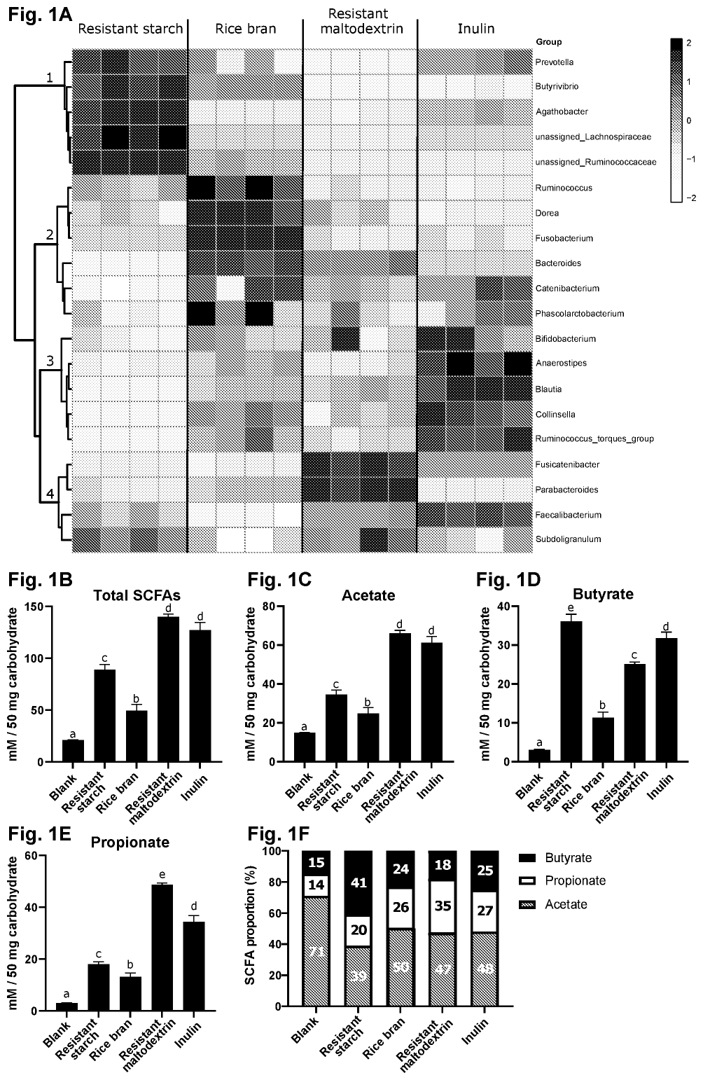

Results: In vitro fecal fermentation of stools obtained from patients with Parkinson's disease (PD) altered the microbiome as evidenced by DNA-based 16S rRNA gene amplicon sequencing. Hierarchical clustering was performed using Euclidean distances and the Ward algorithm, and clusters of taxa were associated with fiber types. Hierarchical clustering of the 25 most abundant genera (heatmap represents log2 relative abundance) after 24 h in vitro fecal fermentation is shown in FIG. 1A. Each fiber enriched specific bacterial taxa, with limited overlap observed between the fibers. Enriched in all four fiber treatments were bacteria with previously demonstrated health-related effects, including bacteria from the genus Prevotella and families Lachnospiraceac and Ruminococaceae (promoted by resistant starch, Cluster 1); genera Ruminoccocus, Dorea, and Bacteroides (promoted by rice bran, Cluster 2); genus Parabacteroides (promoted by resistant maltodextrin, Cluster 3); and genera Faecalibacterium, Anaerostipes and Bifidobacterium (promoted by inulin, Cluster 4) (Zafar et al., Gut microbes 13: 1-20 (2021); Lopetuso et al., Gut Pathog 5: 23 (2013); Tojo et al., World J Gastroenterol 20: 15163-15176 (2014); DeMartino et al., Curr Opin Biotechnol 61: 66-71 (2020); Hiippala et al., “Isolation of Anti-Inflammatory and Epithelium Reinforcing Bacteroides and Parabacteroides Spp. from A Healthy Fecal Donor,” Nutrients 12 (2020); and Wang et al., Cell Rep 26: 222-235 e225 (2019)).

Short chain fatty acid (SCFA) production also increased as shown in FIGS. 1B-1E (bars denoted by a different letter indicate significant differences between treatment means (p<0.05)). The proportions of the SCFA produced during the 24 h in vitro fecal fermentation are shown in FIG. 1F. All fibers promoted SCFA production with resistant maltodextrin and inulin producing the most total SCFA followed by resistant starch and rice bran (FIG. 1B). Moreover, each fiber type had a distinct metabolic signature—resistant starch was highly butyrogenic, resistant maltodextrin was highly propiogenic, and inulin and rice bran promoted production of all three SCFA (FIGS. 1C-F). Although rice bran produced less SCFA (i.e., was less fermentable than the other fibers), it should be noted that rice brain increased the abundance of unique bacteria that were not enriched by the other fibers. Thus, to support the growth of a diverse group of beneficial bacterial groups and promote production of all three SCFA, the following fiber mixture composition was used: 30% resistant starch (raw potato starch), 30% resistant maltodextrin (Nutriose™), 30% stabilized rice bran, and 10% agave branched inulin. These fiber proportions were subsequently incorporated into a highly palatable bar which was given to participants in this study.

### Example 2

### In Vivo Study: Study Design, Participants, and Data/Sample Collection

Participants: All subjects signed the Rush University Medical Center (RUMC) Institutional Review Board approved informed consent form (ORA #: 20072703), and the study was registered (ClinicalTrials.gov Identifier: NCT04512599). The study was an open-label, non-randomized study in PD participants (n=20) at RUMC, including de novo (untreated, n=10) and treated PD participants (n=10) receiving levodopa (LD) and/or other PD medications. A movement disorder neurologist specialist (DAH) examined and confirmed the diagnosis of PD patients. Parkinsonian symptoms were assessed using the Unified Parkinson's Disease Rating Scale (UPDRS) Part 3 (Fahn et al., “Unified Parkinson's Disease Rating Scale,” In: Fahn et al., eds. Recent development in Parkinson's disease. Florhan Park: Macmillan Health Care Information, 153-164 (1987)), and Hoehn and Yahr (H&Y) staging scale (Hoehn et al., Neurology 17: 427-442 (1967)). Participant characteristics are shown in Table 1: age of onset, disease duration, motor UPDRS, H&Y, levodopa daily dosages (LEDD), Bristol stool score, and demographic data (i.e., age, sex race, ethnicity). Inclusion criteria were current diagnosis of PD (UK Brain Bank Criteria, H&Y stages 1-4 inclusive) (Hughes et al., J Neurol Neurosurg Psychiatry 55: 181-184 (1992)), age (>30), and able to consent. Exclusion criteria were: (1) intestinal resection, (2) history of GI disease except for hiatal hernia, GERD, or hemorrhoids, (3) severe renal disease defined by creatinine more than 2½ times normal, (4) markedly abnormal liver function defined by ALT/AST over 4 times normal or elevated bilirubin, (5) antibiotic use within the 12 weeks prior to enrollment, (6) consumption of probiotics, prebiotics, or synbiotics within the 4 weeks prior to enrollment, (7) non-standard diet (e.g., vegan, vegetarian, gluten-free, or Paleo).

TABLE 1
Parkinson's Disease Participant's Demographics

| Demographic and | De Novo PD | | Treated PD | | p- |
|---|---|---|---|---|---|
| Clinical Variables | (n = 10) | | (n = 10) | | value |
| Age, mean (sd) | 62.90 | (6.89) | 65.70 | (9.03) | 0.45 |
| Age of onset, mean (sd) | 61.30 | (6.01) | 59.50 | (9.96) | 0.63 |
| Sex, n (%) | | | | | |
| Men | 5 | (50%) | 6 | (60%) | 1.00 |
| Women | 5 | (50%) | 4 | (40%) | |
| White Race, n (%) | 10 | (100%) | 10 | (100%) | 1.00 |
| Not Hispanic or Latino, n | 10 | (100%) | 10 | (100%) | 1.00 |
| (%) | | | | | |
| Disease duration, mean (sd) | 1.95 | (1.30) | 6.20 | (4.51) | 0.01 |
| H&Y, mean (sd) | 2 | (0) | 2 | (0) | 1.00 |
| UPDRS motor, mean (sd) | 12 | (5.09) | 14.9 | (5.62) | 0.24 |
| LEDD,* median (IQR) | NA | | 393.75 | (333.00) | — |
| Bristol Stool Score, mean | 3.20 | (1.54) | 2.20 | (1.75) | 0.19 |
| (sd) | | | | | |

LEDD*, levodopa equivalent dose; %, percentage; IQR = inter-quartile range, sd, standard deviation; H&Y, Hoehn and Yahr staging scale; UPDRS, Unified Parkinson's Disease Rating Scale.
Bristol Stool Score range (1-7): 1 = severe constipation; 4 = normal; 7 = severe diarrhea.
Independent t-test or chi-square analyses.
Significance: p-value < 0.05 (bold italics).

Design: Each participant had a baseline (BL) visit and a follow up visit after 10 days of the prebiotic intervention. Participants consumed the prebiotics in the form of a bar containing inulin, resistant starch type 2, resistant maltodextrin, and stabilized rice bran prebiotic fibers for 10 days: one bar (10 g fiber) daily for the first three days and then one bar twice a day for an additional week. Ingredients of the bar were organic, generally recognized as safe (GRAS), food-grade ingredients.

Data/Sample Collection: Participants completed the PROMIS questionnaire (Spiegel et al., Am J Gastroenterol 109: 1804-1814 (2014)) to assess the impact of the prebiotic intervention on GI function including bowel movements, stool consistency, discomfort, abdominal pain, bloating, and flatulence on a scale from 1 (best) to 10 (worst). Diet information was collected using the Food Frequency Questionnaire (FFQ), and subjects were asked to continue their usual diet during the 10 days of the study. A Unified Parkinson's Disease Rating Scale (UPDRS) was performed. Participants were asked to collect stool at home 12-24 hours before the BL and end-of-study visits using an anaerobic collection kit (BD Gaspak, Becton Dickinson and Company, Sparks, MD), as previously described (Engen et al., Front Neurol 11: 978 (2020)). Additionally, blood was collected at BL and at the end of the study. Blood was processed for serum and plasma within one hour of collection and was stored at −80° until analysis.

Fecal Microbiome Interrogation: Stool microbiota were assessed using non-targeted shotgun metagenomic sequencing and taxonomic and functional gene profiling. Total DNA was extracted from fecal samples utilizing the FastDNA bead-beating Spin Kit for Soil (MP Biomedicals, Solon, OH, USA) and verified with fluorometric quantitation (Qubit 3.0, Life Technologies, Grand Island, NY). Library preparation was performed using a Swift 2 Turbo DNA Library kit (Swift Biosciences, Ann Arbor, MI) with 50 ng of input DNA and 5 cycles of PCR for indexing with unique dual indices. Libraries were sequenced on an Illumina NovaSeq6000 instrument employing an SP flowcell (paired-end 2×150 base reads). Libraries were created in the Genomics and Microbiome Core Facility at Rush University, and sequencing was performed at the DNA Services Lab at the University of Illinois at Urbana-Champaign.

Sequence reads were quality filtered and trimmed using the algorithm bbduk (Department of Energy Joint Genome Institute) (Andrews, FastQC: a quality control tool for high throughput sequence data [online]). Taxonomic profiling was generated with MetaPh1An3 (v3.0.7) and functional profiling was performed using the software package HUMAnN3 (v3.0.0.α.3) mapping to the UniRef90 catalog (UniRef release 2019_01) (Beghini et al., Elife 10 (2021)). UniRef90 relative abundance tables were regrouped into the following higher-level organizations: MetaCyc pathways, KEGG orthology, and UniProt gene families. Raw sequence data (FASTQ files) were deposited in the National Center for Biotechnology Information (NCBI) Sequence Read Archive (SRA), under the BioProject identifier PRJNA756556.

SCFA Analysis: SCFA analyses were conducted at the Proteomics and Metabolomics Facility of Colorado State University (Fort Collins, CO, USA) using gas chromatography-mass spectrometry (GC-MS). Plasma (200 μL) was added to 30 μL of cold internal standard solution containing 50 μg/mL of ¹³C2-acetate in 2 N HCl. Samples were vortexed (30 s), followed by the addition of 0.35 mL of cold methyl tertiary-butyl ether (MTBE), and were again vortexed (15 s). Samples were centrifuged (3,000 g, 10 min, 4° C.) and the top MTBE layer was recovered and stored at −20° C. until analysis. The MTBE extract (1 μL) was injected into a Trace 1310 GC coupled to a Thermo ISQ-LT MS (Thermo Fisher Scientific, Waltham, MA, USA) at a 5:1 split ratio. The inlet was held at 240° C. SCFA separation was achieved on a 30 m DB-WAXUI column (J&W, 0.25 mm ID, 0.25 μm film thickness). Oven temperature was held at 100° C. for 0.5 min, ramped at 10° C./min to 175° C., then ramped to 240° C. at 40° C./min, and held at 240° C. for 3 min. Helium carrier gas flow was held at 1.2 mL/min. Temperatures of transfer line and ion source were both held at 250° C. SIM mode was used to scan ions 45, 60, 62, 73, 74, 88 at a rate of 10 scans/sec under electron impact mode. Data processing was completed using Chromeleon software (Thermo Fisher Scientific).

Intestinal Inflammation: Fecal calprotectin was used to assess intestinal inflammation (Mulak et al., Front Neurosci 13: 992 (2019)). ELISA was used to examine fecal calprotectin levels according to manufacturer's instructions (BÜHLMANN fCAL® ELISA-EK-CAL; BUHLMANN Diagnostics Corp, Amherst, NH, USA).

Gut Leakiness and Inflammation: Plasma zonulin (a marker of intestinal barrier integrity; MBS706368, MyBioSource) (Fasano, Ann N Y Acad Sci 1258: 25-33 (2012)) and lipopolysaccharide binding protein (LBP, a marker of bacterial translocation; HK315-01, Hycult Biotech) (Gutsmann et al., Infect Immun 69: 6942-6950 (2001)) were assessed via ELISA. Serum inflammatory cytokines were assessed using the V-PLEX Proinflammatory Panel 1 Human Kit (K15049D-1, Meso Scale Diagnostics, LLC, Rockville, MD, USA), including interferon-gamma (IFN-γ), interleukin (IL)-6, IL-8, IL-10, and tumor necrosis factor-alpha (TNF-α). All assays were conducted according to manufacturer's protocol.

Systemic Markers of Brain Health: Serum high mobility group box 1 protein (HMGB-1) was examined as a neuroinflammatory biomarker (ELISA NBP2-62766, NOVUS Biological) (Paudel et al., Front Neurosci 12: 628 (2018)). Serum brain derived neurotrophic factor (BDNF) was assessed as a marker of neuronal health (K1516WK-1, U-PLEX, Meso Scale Diagnostics, LLC, Rockville, MD, USA) (Cohen-Cory et al., Dev Neurobiol 70: 271-288 (2010)). Serum neurofilament light chain (NfL) was evaluated as a biomarker of neurodegenerative disease progression (F217X-3, R-PLEX, Meso Scale Diagnostics, LLC, Rockville, MD, USA) (Bacioglu et al., Neuron 91: 494-496 (2016)).

Statistical Analysis: Demographics, including age, sex, treatment status, H& Y stage, and duration of disease were described using descriptive statistics. The primary outcomes for this pilot study were feasibility of consuming the bar, GI symptoms, changes in the blood marker of intestinal barrier, and alterations in the relative abundance of fecal SCFA-producing bacteria.

Changes in GI symptoms and side effects (bloating, diarrhea) from BL to post bar and between the two groups were tested with a paired t-test or Wilcoxon signed rank test. Regression analysis was performed for changes in GI symptoms with group, age, duration of disease and levodopa equivalent dose in the model. Exploratory analyses were used to compare the UPDRS motor scores between BL and post with a paired t-test. A p-value<0.05 was considered significant. No adjustment for multiple testing was applied to this pilot study.

Blood markers of gut leakiness, systemic inflammation, brain health and SCFA metabolites, as well as fecal calprotectin were analyzed using the Wilcoxon-signed rank test for pairwise group comparisons. Analyses were conducted using the software GraphPad Prism (v9.0, GraphPad Software LLC San Diego California).

Analyses of alpha-and beta-diversity were used to examine changes in fecal microbial community structure. Alpha-diversity metrics (i.e., Shannon index, Simpson's index, richness and evenness) were calculated from rarefied datasets (1 million sequences/sample). Group comparisons were performed with Wilcoxon-signed rank paired test in GraphPad Prism (v9.0). Permutation Multivariate Analysis of Variance (PERMANOVA) (Kelly et al., Bioinformatics 31: 2461-2468 (2015)) was utilized to compare fecal microbial community structure before and after prebiotic intervention. Significance of PERMANOVA values were determined using 9,999 permutations and corrected for multiple testing using the Benjamini-Hochberg method (q<0.05). Paired Bray-Curtis dissimilarity based non-multidimensional scaling (nMDS) plots of the bacterial species community were used to visualize the PD participant's BL and prebiotic individual samples. Each sample was connected to a centroid representing the mean value of the group (i.e., BL or Prebiotic). The Wilcoxon-signed rank test was used to correct for multiple testing and identify significant differences in the relative abundance of taxa (e.g., phylum and species) and functional gene/pathways for paired analyses before and after the prebiotic intervention. Spearman's rank correlation coefficients were generated between the relative abundances of species, experimental measures, and clinical parameters.

### Results

a. Tolerability and Feasibility of Prebiotic Bar Consumption by PD Patients

Participants with PD consumed the prebiotic bar for 10 days. Consumption of prebiotics can impact GI symptoms, thus acceptability, tolerability, and impact on GI function were assessed. Subjects were asked how likely they were to continue eating the prebiotic bars daily with a score ranging from 0 (not likely) to 10 (highly likely). Nearly all participants reported that they would be highly likely to continue eating the prebiotic bar, (Table 2) indicating that the bar was both well-tolerated and palatable.

TABLE 2
Primary Outcomes: Tolerability and Gastrointestinal Symptoms
Tolerability.

| | De Novo PD | | Treated PD | |
|---|---|---|---|---|
| | (n = 10) | | (n = 10) | |
| Would continue the bar, median | 9.5 | (5) | 8 | (5) |
| (IQR) | | | | |
| Ease of eating 1 bar, median (IQR) | 9 | (4) | 10 | (1) |
| Ease of eating 2 bars, median (IQR) | 7.5 | (4) | 8 | (2) |
| Ease of eating 3 bars, median (IQR) | 4 | (6) | 5 | (4) |

Gastrointestinal Symptoms.

| | | |
|---|---|---|
| Baseline | Prebiotic | p-value |

All PD (n = 20)

| | | | | | |
|---|---|---|---|---|---|
| Total score*, mean (sd) | 35.85 | (25.91) | 27.65 | (23.16) | 0.09 |
| Constipation, median (IQR) | 0.5 | (5) | 0 | (2) | 0.29 |
| Infrequent bowel movements, | 1.5 | (5) | 1 | (2) | 0.20 |
| median (IQR) | | | | | |

De Novo PD (n = 10)

| | | | | | |
|---|---|---|---|---|---|
| Total score, mean (sd) | 29.90 | (26.87) | 32.60 | (30.18) | 0.63 |
| Constipation, median (IQR) | 0 | (2) | 0 | (2) | 0.50 |
| Infrequent bowel movements, | 0.5 | (2) | 1 | (1) | 0.94 |
| median (IQR) | | | | | |

Treated PD (n = 10)

| | | | | | |
|---|---|---|---|---|---|
| Total score, mean (sd) | 41.8 | (24.82) | 22.7 | (12.91) | 0.01 |
| Constipation, median (IQR) | 4 | (7) | 0.5 | (2) | 0.09 |
| Infrequent bowel movements, | 3.5 | (7) | 1 | (2) | 0.06 |
| median (IQR) | | | | | |

Data are shown as median (interquartile range) or mean (standard deviation).
IQR = inter-quartile range, sd, standard deviation.
Significance: p-value < 0.05 (bold italics).
Gastrointestinal evaluation used the PROMIS* gastrointestinal symptom scale to examine GI symptoms and severity.
(*= Spiegel et al., Am J Gastroenterol 109: 1804-1814 (2014)).

GI symptoms were compared from BL to after the intervention in both de novo and treated PD participants (Table 2). Bloating and diarrhea at BL and after the intervention were similar in de novo (p=0.45 and p=1.0) and treated participants (p=0.50 and p=1.0) indicating that the prebiotic bar did not cause/promote negative GI side effects. Comparing the GI Symptom and Severity Checklist score revealed that treated PD participants had a significant improvement in the total GI score after the prebiotic intervention (p=0.01), an effect that was not observed in de novo participants. Regression analyses of the total GI symptom score (controlling for age, duration of disease, and levodopa equivalent dose) revealed a significant difference between de novo and treated PD participants in the total GI Symptom and Severity Checklist score from BL to post bar (improved after the intervention, p=0.04), but no difference in constipation (p=0.23) nor frequency of bowel movements (p=0.54) (data not shown). Taken together, these results indicate that were no side effects associated with consumption of the prebiotic fiber mixture, and GI symptoms improved in treated PD participants. Consumption of the prebiotic bar for 10 days was associated with a significant decrease in total UPDRS score from BL (median 11.50, IQR 8.0) to after the prebiotic intervention (median 9.0, IQR 6.5; p<0.01).

b. Prebiotic Fiber Mixture Changed the Microbiome

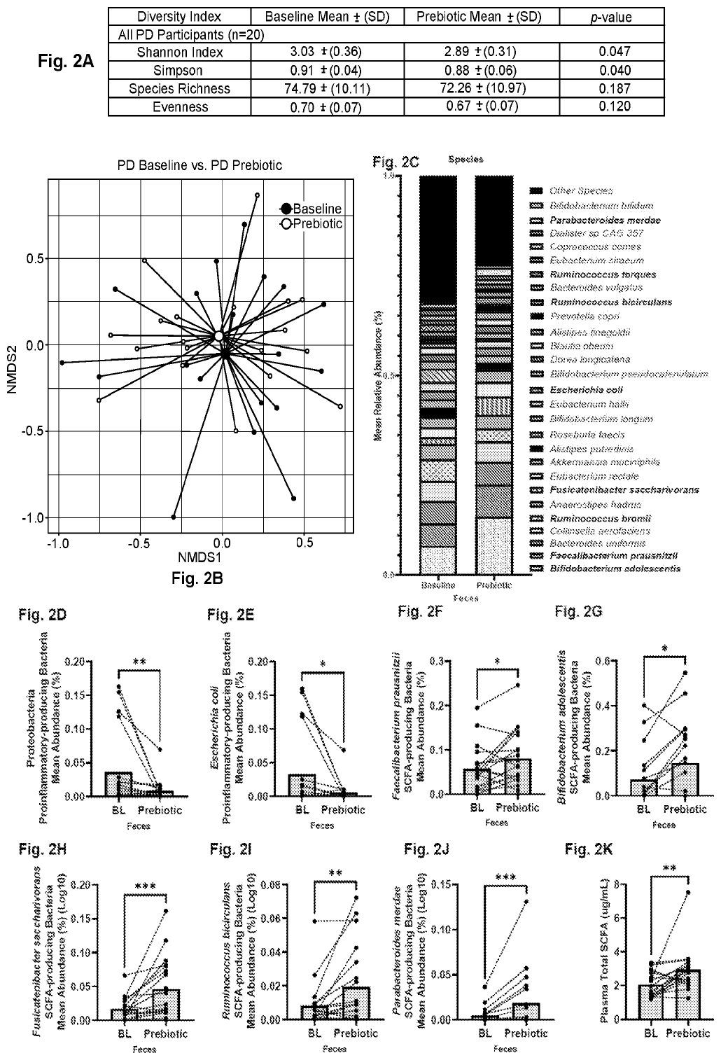

The prebiotic intervention increased levels of SCFA in the plasma (FIG. 2K; Wilcoxon signed rank pair test, *p<0.05; **p<0.01; ***p<0.001).

There were no significant differences in the microbiome of de novo and treated PD participants at BL (PERMANOVA: q=0.280); thus, these groups were combined for subsequent analyses. Diversity indices of Shannon Index, Simpson's Index, Species Richness and Pielou's evenness were measured at the taxonomic level of species. Alpha diversity metrics (Shannon index and Simpson) significantly decreased after the prebiotic intervention (FIG. 2A; mean index score and standard deviation (SD) are displayed). Although differences in alpha diversity were noted, total microbial community structure was not significantly impacted by the prebiotic intervention (PERMANOVA: q=0.425; FIG. 2B; centroids for each group representing the mean value of the baseline (red) or prebiotic (green) groups are shown; non-metric MDS plots were built on Bray-Curtis dissimilarity metrics). Nonetheless, the prebiotic intervention was associated with differences in taxon abundances at the species level (FIG. 2C, which shows the mean relative abundance of taxa with greater than 1% average relative abundance (n=20); differentially abundant taxa are bolded (Wilcoxon signed rank pair-test: p<0.05); Table 3)). The relative abundance of putative pro-inflammatory groups, including the phylum Protcobacteria (q=0.016) and species Escherichia coli (p=0.031), were significantly lower after the prebiotic intervention relative to BL (FIGS. 2D-2E). Conversely, the relative abundance of putative SCFA-producing species, such as Faecalibacterium prausnitizii, Bifidobacterium adolescentis, Ruminococcus bicirculans, Fusicatenibacter saccharivorans, and Parabacteroides merdae, were significantly (p<0.05) higher after the prebiotic intervention relative to BL (FIGS. 2F-2J). These prebiotic-induced changes in the bacterial taxa were accompanied by significant changes (q<0.05) in the abundances of 64 genomic pathways (Table 4), indicating an overall change in the functional capacity of the microbiota. It is noteworthy that prebiotic treatment downregulated multiple biosynthetic pathways (Table 4) that were reported to be upregulated in PD patients when compared to their spouse's fecal samples (Mao et al., Front Microbiol 12: 728479 (2021)). In particular, the acetyl-CoA fermentation to butanoate II (PWY-5676) was enriched in PD subjects relative to their spouses but was downregulated post prebiotic in all PD subjects that we studied (Table 4). The prebiotic intervention-induced changes in the microbiota were associated with a concurrent increase in plasma total SCFA (p=0.006) in participants (FIG. 2K), and each individual SCFA: acetate (p=0.006), propionate (p=0.006), butyrate (p=0.059) and the (total propionate+butyrate):(total SCFA) ratio (p=0.030) (Table 5). Subgroup analysis of de novo and treated PD participants were also analyzed separately (Tables 6 and 7).

TABLE 3
Relative abundances of bacterial taxa altered between all PD
participants before and after prebiotic consumption samples.

| | | | |
|---|---|---|---|
| | Baseline | Prebiotic | |
| | Mean RA % ± | Mean RA % ± | q or p |
| Taxonomic Level | (SD) % | (SD) % | values |

Phylum

| | | | |
|---|---|---|---|
| Firmicutes | 51.08 ± (20.93) | 47.09 ± (18.48) | 0.395 |
| Actinobacteria | 21.29 ± (18.97) | 25.21 ± (21.13) | 0.293 |
| Bacteroidetes | 21.27 ± (17.40) | 23.79 ± (14.89) | 0.418 |
| Verrucomicrobia | 2.71 ± (4.65) | 3.01 ± (6.01) | 0.783 |
| Proteobacteria | 3.63 ± (5.65) | 0.86 ± (1.58) | 0.016 |

Phylum: Species

| | | | |
|---|---|---|---|
| Actinobacteria: Bifidobacterium adolescentis | 7.28 ± (12.19) | 14.63 ± (17.03) | 0.018 |
| Firmicutes: Faecalibacterium prausnitzii | 5.72 ± (5.17) | 8.07 ± (6.22) | 0.049 |
| Bacteroidetes: Bacteroides uniformis | 5.42 ± (7.26) | 5.65 ± (5.70) | 0.896 |
| Actinobacteria: Collinsella aerofaciens | 5.20 ± (3.67) | 5.05 ± (4.15) | 0.930 |
| Firmicutes: Ruminococcus bromii | 5.38 ± (7.78) | 3.25 ± (4.23) | 0.019 |
| Firmicutes: Anaerostipes hadrus | 3.78 ± (4.06) | 3.43 ± (2.28) | 0.921 |
| Firmicutes: Fusicatenibacter saccharivorans | 1.65 ± (1.65) | 4.61 ± (4.45) | 0.021 |
| Firmicutes: Eubacterium rectale | 2.50 ± (2.71) | 3.69 ± (5.30) | 0.410 |
| Verrucomicrobia: Akkermansia muciniphila | 2.71 ± (4.65) | 3.01 ± (6.01) | 0.783 |
| Bacteroidetes: Alistipes putredinis | 2.28 ± (2.71) | 1.80 ± (2.25) | 0.530 |
| Firmicutes: Roseburia faecis | 2.03 ± (3.32) | 1.98 ± (3.76) | 0.949 |
| Actinobacteria: Bifidobacterium longum | 2.23 ± (3.65) | 1.76 ± (2.94) | 0.484 |
| Firmicutes: Eubacterium hallii | 2.18 ± (2.69) | 1.79 ± (1.59) | 0.887 |
| Proteobacteria: Escherichia coli | 3.29 ± (5.72) | 0.58 ± (1.56) | 0.031 |
| Actinobacteria: Bifidobacterium pseudocatenulatum | 2.14 ± (3.31) | 1.48 ± (3.29) | 0.155 |
| Firmicutes: Dorea longicatena | 1.63 ± (1.70) | 1.88 ± (1.53) | 0.206 |
| Firmicutes: Blautia obeum | 1.75 ± (2.58) | 1.46 ± (1.89) | 0.930 |
| Bacteroidetes: Alistipes finegoldii | 1.03 ± (1.98) | 1.88 ± (2.79) | 0.185 |
| Bacteroidetes: Prevotella copri | 0.92 ± (2.54) | 1.81 ± (7.45) | 1.00 |
| Firmicutes: Ruminococcus bicirculans | 0.79 ± (1.34) | 1.91 ± (2.37) | 0.009 |
| Bacteroidetes: Bacteroides vulgatus | 1.25 ± (1.77) | 1.32 ± (1.80) | 0.443 |
| Firmicutes: Ruminococcus torques | 1.64 ± (2.16) | 0.70 ± (0.84) | 0.024 |
| Firmicutes: Eubacterium siraeum | 1.09 ± (4.11) | 1.22 ± (2.93) | 0.069 |
| Firmicutes: Coprococcus comes | 1.23 ± (1.54) | 1.08 ± (1.53) | 0.513 |
| Firmicutes: Dialister sp. CAG. 357 | 1.33 ± (3.38) | 0.95 ± (2.35) | 1.000 |
| Bacteroidetes: Parabacteroides merdae | 0.45 ± (0.91) | 1.82 ± (3.28) | 0.044 |
| Actinobacteria: Bifidobacterium bifidum | 1.16 ± (3.33) | 0.88 ± (2.54) | 1.000 |

Mean RA % = average number of sequences per taxa, calculated from the total sum of all sequence counts, depicted as a percentage.
Microbial taxa (>1%) shown.
(SD) % = standard deviation as a percentage.
Wilcoxon signed-rank paired test used to compare two related samples for each PD participant's group.
Benjamini-Hochberg significance: q-value < 0.05 (grey/bold italics); p-value < 0.05 (bold italics).
All PD baseline and prebiotic (n = 20 per group).

TABLE 4
The differential abundances of functional gene pathways
that were significantly downregulated between all PD participants
before and after prebiotic consumption samples.

| | | | Baseline | | |
|---|---|---|---|---|---|
| Significant Functional | | | Mean | Prebiotic Mean | |
| Gene Pathways | p value | q value | Abundance | Abundance | Log2FC |
| P164-PWY: purine nucleobases | 0.001 | 0.017 | 607.65 | 487.61 | −0.32 |
| degradation I (anaerobic) | | | | | |
| TEICHOICACID-PWY: | 0.001 | 0.017 | 414.14 | 320.13 | −0.37 |
| teichoic acid (poly-glycerol) | | | | | |
| biosynthesis | | | | | |
| PWY-7210: pyrimidine | 0.000 | 0.017 | 844.62 | 615.23 | −0.46 |
| deoxyribonucleotides | | | | | |
| biosynthesis from CTP | | | | | |
| PWY-5188: tetrapyrrole | 0.001 | 0.017 | 1272.45 | 921.54 | −0.47 |
| biosynthesis I (from glutamate) | | | | | |
| ASPASN-PWY: superpathway | 0.000 | 0.017 | 1515.34 | 1043.34 | −0.54 |
| of L-aspartate and L-asparagine | | | | | |
| biosynthesis | | | | | |
| PWY-5104: L-isoleucine | 0.000 | 0.017 | 1871.58 | 1276.48 | −0.55 |
| biosynthesis IV | | | | | |
| PWY-6608: guanosine | 0.001 | 0.017 | 1386.07 | 884.77 | −0.65 |
| nucleotides degradation III | | | | | |
| PWY66-409: superpathway of | 0.001 | 0.017 | 996.50 | 488.10 | −1.03 |
| purine nucleotide salvage | | | | | |
| PWY-4981: L-proline | 0.001 | 0.017 | 684.93 | 328.69 | −1.06 |
| biosynthesis II (from arginine) | | | | | |
| PWY0-1479: tRNA processing | 0.000 | 0.017 | 720.80 | 281.93 | −1.35 |
| MET-SAM-PWY: | 0.001 | 0.019 | 1580.85 | 1098.93 | −0.52 |
| superpathway of S-adenosyl-L- | | | | | |
| methionine biosynthesis | | | | | |
| PWY-7211: superpathway of | 0.002 | 0.019 | 1144.49 | 879.95 | −0.38 |
| pyrimidine | | | | | |
| deoxyribonucleotides de novo | | | | | |
| biosynthesis | | | | | |
| PWY-6545: pyrimidine | 0.001 | 0.019 | 951.92 | 729.06 | −0.38 |
| deoxyribonucleotides de novo | | | | | |
| biosynthesis III | | | | | |
| PWY-7220: adenosine | 0.002 | 0.019 | 2598.75 | 1932.85 | −0.43 |
| deoxyribonucleotides de novo | | | | | |
| biosynthesis II | | | | | |
| PWY-7222: guanosine | 0.002 | 0.019 | 2598.75 | 1932.85 | −0.43 |
| deoxyribonucleotides de novo | | | | | |
| biosynthesis II | | | | | |
| PWY-5347: superpathway of L- | 0.001 | 0.019 | 1485.71 | 1022.12 | −0.54 |
| methionine biosynthesis | | | | | |
| (transsulfuration) | | | | | |
| METSYN-PWY: L-homoserine | 0.001 | 0.019 | 1492.30 | 1013.20 | −0.56 |
| and L-methionine biosynthesis | | | | | |
| HOMOSER-METSYN-PWY: | 0.002 | 0.019 | 957.68 | 619.77 | −0.63 |
| L-methionine biosynthesis I | | | | | |
| FUC-RHAMCAT-PWY: | 0.002 | 0.019 | 486.61 | 281.12 | −0.79 |
| superpathway of fucose and | | | | | |
| rhamnose degradation | | | | | |
| PWY-6628: superpathway of L- | 0.002 | 0.019 | 652.12 | 229.53 | −1.51 |
| phenylalanine biosynthesis | | | | | |
| PWY-841: superpathway of | 0.002 | 0.021 | 1839.62 | 1419.98 | −0.37 |
| purine nucleotides de novo | | | | | |
| biosynthesis I | | | | | |
| PWY-6892: thiazole | 0.002 | 0.022 | 2181.98 | 1744.11 | −0.32 |
| biosynthesis I (E. coli) | | | | | |
| ARG-POLYAMINE-SYN: | 0.002 | 0.022 | 802.88 | 477.74 | −0.75 |
| superpathway of arginine and | | | | | |
| polyamine biosynthesis | | | | | |
| POLYAMSYN-PWY: | 0.002 | 0.022 | 508.71 | 281.69 | −0.85 |
| superpathway of polyamine | | | | | |
| biosynthesis I | | | | | |
| PWY-7184: pyrimidine | 0.003 | 0.025 | 1132.29 | 798.22 | −0.50 |
| deoxyribonucleotides de novo | | | | | |
| biosynthesis I | | | | | |
| PWY0-166: superpathway of | 0.003 | 0.027 | 1555.16 | 1202.56 | −0.37 |
| pyrimidine | | | | | |
| deoxyribonucleotides de novo | | | | | |
| biosynthesis (E. coli) | | | | | |
| PWY-7208: superpathway of | 0.003 | 0.027 | 1097.81 | 821.67 | −0.42 |
| pyrimidine nucleobases salvage | | | | | |
| DENOVOPURINE2-PWY: | 0.004 | 0.028 | 1918.89 | 1520.45 | −0.34 |
| superpathway of purine | | | | | |
| nucleotides de novo | | | | | |
| biosynthesis II | | | | | |
| PWY-6125: superpathway of | 0.004 | 0.028 | 1510.98 | 1117.02 | −0.44 |
| guanosine nucleotides de novo | | | | | |
| biosynthesis II | | | | | |
| PWY0-1297: superpathway of | 0.004 | 0.028 | 770.24 | 425.16 | −0.86 |
| purine deoxyribonucleosides | | | | | |
| degradation | | | | | |
| PWY-7187: pyrimidine | 0.005 | 0.029 | 1549.82 | 1216.08 | −0.35 |
| deoxyribonucleotides de novo | | | | | |
| biosynthesis II | | | | | |
| PWY-2941: L-lysine | 0.005 | 0.029 | 940.31 | 675.77 | −0.48 |
| biosynthesis II | | | | | |
| PWY-5345: superpathway of L- | 0.005 | 0.029 | 794.65 | 345.74 | −1.20 |
| methionine biosynthesis (by | | | | | |
| sulfhydrylation) | | | | | |
| SULFATE-CYS-PWY: | 0.005 | 0.029 | 745.62 | 294.23 | −1.34 |
| superpathway of sulfate | | | | | |
| assimilation and cysteine | | | | | |
| biosynthesis | | | | | |
| PWY-7198: pyrimidine | 0.005 | 0.030 | 907.03 | 693.68 | −0.39 |
| deoxyribonucleotides de novo | | | | | |
| biosynthesis IV | | | | | |
| PWY-7228: superpathway of | 0.005 | 0.030 | 1420.60 | 1036.25 | −0.46 |
| guanosine nucleotides de novo | | | | | |
| biosynthesis I | | | | | |
| PWY4LZ-257: superpathway of | 0.005 | 0.030 | 580.58 | 279.26 | −1.06 |
| fermentation (Chlamydomonas | | | | | |
| reinhardtii) | | | | | |
| P161-PWY: acetylene | 0.005 | 0.030 | 536.12 | 242.82 | −1.14 |
| degradation | | | | | |
| PWY-6126: superpathway of | 0.006 | 0.033 | 3056.38 | 2491.45 | −0.29 |
| adenosine nucleotides de novo | | | | | |
| biosynthesis II | | | | | |
| SALVADEHYPOX-PWY: | 0.006 | 0.033 | 1175.87 | 729.95 | −0.69 |
| adenosine nucleotides | | | | | |
| degradation II | | | | | |
| GLUCOSE1PMETAB-PWY: | 0.006 | 0.033 | 522.38 | 241.36 | −1.11 |
| glucose and glucose-1- | | | | | |
| phosphate degradation | | | | | |
| PWY-1042: glycolysis IV | 0.008 | 0.035 | 4347.25 | 3861.78 | −0.17 |
| (plant cytosol) | | | | | |
| TRPSYN-PWY: L-tryptophan | 0.008 | 0.035 | 2113.63 | 1806.04 | −0.23 |
| biosynthesis | | | | | |
| PWY-7229: superpathway of | 0.008 | 0.035 | 3260.36 | 2705.79 | −0.27 |
| adenosine nucleotides de novo | | | | | |
| biosynthesis I | | | | | |
| PWY-7197: pyrimidine | 0.008 | 0.035 | 764.77 | 550.03 | −0.48 |
| deoxyribonucleotide | | | | | |
| phosphorylation | | | | | |
| HEXITOLDEGSUPER-PWY: | 0.008 | 0.035 | 1330.33 | 924.41 | −0.53 |
| superpathway of hexitol | | | | | |
| degradation (bacteria) | | | | | |
| PWY-5676: acetyl-CoA | 0.007 | 0.035 | 607.71 | 421.37 | −0.53 |
| fermentation to butanoate II | | | | | |
| PWY-6606: guanosine | 0.007 | 0.035 | 691.52 | 405.20 | −0.77 |
| nucleotides degradation II | | | | | |
| PWY-7328: superpathway of | 0.007 | 0.035 | 508.07 | 253.94 | −1.00 |
| UDP-glucose-derived O- | | | | | |
| antigen building blocks | | | | | |
| biosynthesis | | | | | |
| PWY-6113: superpathway of | 0.008 | 0.035 | 709.66 | 279.59 | −1.34 |
| mycolate biosynthesis | | | | | |
| GLYCOLYSIS-TCA- | 0.008 | 0.035 | 586.22 | 145.15 | −2.01 |
| GLYOX-BYPASS: | | | | | |
| superpathway of glycolysis, | | | | | |
| pyruvate dehydrogenase, | | | | | |
| TCA, and glyoxylate bypass | | | | | |
| GLUTORN-PWY: L- | 0.011 | 0.044 | 2920.17 | 2581.73 | −0.18 |
| ornithine biosynthesis | | | | | |
| PWY66-422: D-galactose | 0.011 | 0.044 | 2483.03 | 2146.14 | −0.21 |
| degradation V (Leloir | | | | | |
| pathway) | | | | | |
| PWY-6353: purine | 0.011 | 0.044 | 1119.04 | 723.09 | −0.63 |
| nucleotides degradation II | | | | | |
| (aerobic) | | | | | |
| PWY-7111: pyruvate | 0.012 | 0.045 | 4542.19 | 3930.69 | −0.21 |
| fermentation to isobutanol | | | | | |
| (engineered) | | | | | |
| PWY-5667: CDP- | 0.012 | 0.045 | 2794.61 | 2356.61 | −0.25 |
| diacylglycerol biosynthesis I | | | | | |
| PWY0-1319: CDP- | 0.012 | 0.045 | 2794.61 | 2356.61 | −0.25 |
| diacylglycerol biosynthesis | | | | | |
| II | | | | | |
| PWY-6703: preQ0 | 0.012 | 0.045 | 1585.87 | 1293.57 | −0.29 |
| biosynthesis | | | | | |
| PWY0-781: aspartate | 0.012 | 0.045 | 1078.46 | 737.51 | −0.55 |
| superpathway | | | | | |
| FAO-PWY: fatty acid | 0.012 | 0.045 | 593.82 | 201.47 | −1.56 |
| &beta;- oxidation I | | | | | |
| PWY0-1586: peptidoglycan | 0.014 | 0.048 | 2773.53 | 2142.53 | −0.37 |
| maturation (meso- | | | | | |
| diaminopimelate containing) | | | | | |
| PRPP-PWY: superpathway | 0.014 | 0.048 | 1363.95 | 1037.23 | −0.40 |
| of histidine, purine, and | | | | | |
| pyrimidine biosynthesis | | | | | |
| PWY-621: sucrose | 0.014 | 0.048 | 1113.73 | 787.71 | −0.50 |
| degradation III (sucrose | | | | | |
| invertase) | | | | | |
| PWY-5971: palmitate | 0.014 | 0.048 | 903.19 | 441.24 | −1.03 |
| biosynthesis II (bacteria and | | | | | |
| plants) | | | | | |

Wilcoxon signed-rank paired test used to compare two related samples for each group.
Benjamini-Hochberg significance: q-value < 0.05 (grey/bold italics); p-value < 0.05 (bold italics).
All PD participants baseline and prebiotic (n = 20 per group).
Mean Abundance = average number of sequences in defined group.
Log2 fold change calculated for each time point.
Pathway abundance data was not rarefied but rather filtered at 0.01% threshold.
Any pathway that had an overall abundance of at least 0.01% of the total abundance was retained for analysis.

TABLE 5
Blood markers of short chain fatty acids metabolites, gut leakiness,
systemic inflammation, brain health, with fecal intestinal inflammation,
between all PD baseline and prebiotic samples

| | | |
|---|---|---|
| Baseline | Prebiotic | p-value |

All PD Participants (n = 20)
Short Chain Fatty Acids - Plasma (ug/mL)

| | | | | | |
|---|---|---|---|---|---|
| Acetate | 1.90 | (0.69) | 2.72 | (1.28) | 0.006 |
| Propionate | 0.08 | (0.04) | 0.12 | (0.05) | 0.006 |
| Butyrate | 0.06 | (0.04) | 0.08 | (0.03) | 0.059 |
| Total SCFA | 2.05 | (0.74) | 2.92 | (1.30) | 0.006 |
| Butyrate-to-Total SCFA Ratio | 0.03 | (0.02) | 0.03 | (0.02) | 0.442 |
| Propionate + Butyrate-to-Total SCFA Ratio | 0.11 | (0.05) | 0.15 | (0.05) | 0.030 |

Intestinal Barrier Integrity & Bacterial Translocation - Plasma (ng/ml)

| | | | | | |
|---|---|---|---|---|---|
| Zonulin | 21.08 | (6.623) | 13.97 | (4.895) | 0.001 |
| LBP | 14,476 | (5,904) | 15,205 | (6621) | 0.277 |

Intestinal Inflammation - Feces (ug/g)

| | | | | | |
|---|---|---|---|---|---|
| Calprotectin | 74.45 | (109) | 54.95 | (86.59) | 0.044 |

Systemic Inflammation - Serum (pg/ml)

| | | | | | |
|---|---|---|---|---|---|
| IFN-γ | 6.38 | (14.02) | 3.36 | (2.43) | 0.294 |
| IL-6 | 0.66 | (0.34) | 0.69 | (0.46) | 0.840 |
| IL-8 | 26.72 | (75.49) | 10.02 | (3.11) | 0.869 |
| IL-10 | 0.28 | (0.21) | 0.33 | (0.32) | 0.216 |
| TNF-α | 0.67 | (0.20) | 0.68 | (0.22) | 0.856 |

Brain Health - Serum (pg/ml) or (ng/ml)

| | | | | | |
|---|---|---|---|---|---|
| BDNF | 5,896 | (1,801) | 5.461 | (1,941) | 0.397 |
| NfL | 70.02 | (35.09) | 63.15 | (33.35) | 0.003 |
| HMGB-1 | 223.5 | (106.8) | 209.4 | (82.20) | 0.756 |

All data are shown as mean (standard deviation).
Wilcoxon signed-rank paired test used to compare two related samples for each PD participant.
Significance: p-value < 0.05 (bold italics).
sd, standard deviation; LBP, lipopolysaccharide binding protein; IFN-γ, interferon-gamma; IL, interleukin; TNF-α, tumor necrosis factor-alpha; BDNF, brain derived-neurotrophic-factor; NfL, neurofilament light chain; HMGB-1, high mobility group box 1 protein.

TABLE 6
Alpha diversity indices and relative abundances of bacterial
taxa alterations between de novo PD or treated PD participants
before and after prebiotic consumption samples.

| | | | |
|---|---|---|---|
| Taxonomic Level | Baseline | Prebiotic | p values |

Diversity Index

| | | | |
|---|---|---|---|
| Shannon Index | 3.10 ± (0.30) | 3.01 ± (0.24) | 0.217 |
| Simpson | 0.92 ± (0.03) | 0.90 ± (0.05) | 0.108 |
| Species Richness | 77.50 ± (7.66) | 74.80 ± (11.75) | 0.326 |
| Evenness | 0.71 ± (0.06) | 0.70 ± (0.05) | 0.383 |

Phylum

| | | | |
|---|---|---|---|
| Firmicutes | 60.39 ± (18.15) | 53.69 ± (16.58) | 0.322 |
| Actinobacteria | 16.24 ± (19.92) | 19.23 ± (16.45) | 0.492 |
| Bacteroidetes | 18.58 ± (16.77) | 23.96 ± (13.00) | 0.431 |
| Verrucomicrobia | 1.90 ± (2.96) | 2.21 ± (3.82) | 1.000 |
| Proteobacteria | 2.87 ± (4.96) | 0.91 ± (2.16) | 0.083 |

Phylum: Species

| | | | |
|---|---|---|---|
| Actinobacteria: Bifidobacterium adolescentis | 3.43 ± (10.28) | 10.36 ± (15.79) | 0.100 |
| Firmicutes: Faecalibacterium prausnitzii | 6.84 ± (6.30) | 9.55 ± (6.76) | 0.064 |
| Bacteroidetes: Bacteroides uniformis | 4.11 ± (5.28) | 5.78 ± (5.40) | 0.375 |
| Actinobacteria: Collinsella aerofaciens | 6.07 ± (3.50) | 5.62 ± (3.42) | 1.000 |
| Firmicutes: Ruminococcus bromii | 6.92 ± (9.84) | 3.99 ± (4.79) | 0.105 |
| Firmicutes: Anaerostipes hadrus | 3.05 ± (3.57) | 3.11 ± (2.33) | 0.769 |
| Firmicutes: Fusicatenibacter saccharivorans | 1.89 ± (1.29) | 5.20 ± (4.86) | 0.009 |
| Firmicutes: Eubacterium rectale | 2.46 ± (2.77) | 2.72 ± (3.09) | 0.726 |
| Verrucomicrobia: Akkermansia muciniphila | 1.90 ± (2.96) | 2.21 ± (3.82) | 1.000 |
| Bacteroidetes: Alistipes putredinis | 3.23 ± (2.80) | 2.02 ± (1.58) | 0.192 |
| Firmicutes: Roseburia faecis | 2.57 ± (4.07) | 2.61 ± (4.27) | 0.528 |
| Actinobacteria: Bifidobacterium longum | 2.25 ± (4.57) | 0.97 ± (1.29) | 0.529 |
| Firmicutes: Eubacterium hallii | 2.21 ± (3.48) | 1.65 ± (1.23) | 1.000 |
| Proteobacteria: Escherichia coli | 2.67 ± (4.96) | 0.81 ± (2.16) | 0.141 |
| Actinobacteria: Bifidobacterium | 1.25 ± (2.61) | 0.45 ± (0.94) | 0.201 |
| pseudocatenulatum | | | |
| Firmicutes: Dorea longicatena | 2.45 ± (1.92) | 2.62 ± (1.41) | 0.695 |
| Firmicutes: Blautia obeum | 2.07 ± (1.47) | 2.21 ± (2.33) | 1.000 |
| Bacteroidetes: Alistipes finegoldii | 0.65 ± (1.62) | 0.97 ± (1.42) | 0.624 |
| Bacteroidetes: Prevotella copri | 1.61 ± (3.40) | 3.44 ± (10.24) | 1.000 |
| Firmicutes: Ruminococcus bicirculans | 1.25 ± (1.80) | 2.94 ± (2.66) | 0.014 |
| Bacteroidetes: Bacteroides vulgatus | 0.99 ± (1.55) | 1.05 ± (1.42) | 0.183 |
| Firmicutes: Ruminococcus torques | 2.31 ± (2.43) | 0.81 ± (0.84) | 0.024 |
| Firmicutes: Eubacterium siraeum | 0.21 ± (0.28) | 0.80 ± (1.02) | 0.042 |
| Firmicutes: Coprococcus comes | 1.54 ± (1.96) | 1.21 ± (1.96) | 0.769 |
| Firmicutes: Dialister sp. CAG. 357 | 1.23 ± (3.91) | 0.92 ± (2.92) | 1.000 |
| Bacteroidetes: Parabacteroides merdae | 0.45 ± (0.63) | 1.79 ± (2.16) | 0.014 |
| Actinobacteria: Bifidobacterium bifidum | 0.77 ± (2.38) | 0.23 ± (0.55) | 1.000 |

Treated PD Participants (n = 10)

| | | | |
|---|---|---|---|
| Diversity Index | | | |
| Shannon Index | 2.96 ± (0.41) | 2.76 ± (0.35) | 0.134 |
| Simpson | 0.90 ± (0.05) | 0.87 ± (0.07) | 0.233 |
| Species Richness | 71.78 ± (12.02) | 69.44 ± (9.92) | 0.424 |
| Evenness | 0.69 ± (0.08) | 0.65 ± (0.09) | 0.214 |

Phylum

| | | | |
|---|---|---|---|
| Firmicutes | 40.73 ± (19.65) | 39.76 ± (18.55) | 0.910 |
| Actinobacteria | 26.90 ± (17.20) | 31.87 ± (24.60) | 0.496 |
| Bacteroidetes | 24.25 ± (18.60) | 23.64 ± (17.57) | 0.910 |
| Verrucomicrobia | 3.60 ± (6.09) | 3.90 ± (7.95) | 0.932 |
| Proteobacteria | 4.48 ± (6.53) | 0.80 ± (0.59) | 0.027 |

Phylum: Species

| | | | |
|---|---|---|---|
| Actinobacteria: Bifidobacterium adolescentis | 11.56 ± (13.28) | 19.38 ± (18.00) | 0.108 |
| Firmicutes: Faecalibacterium prausnitzii | 4.47 ± (3.50) | 6.42 ± (5.45) | 0.496 |
| Bacteroidetes: Bacteroides uniformis | 6.88 ± (9.09) | 5.05 ± (6.34) | 0.441 |
| Actinobacteria: Collinsella aerofaciens | 4.24 ± (3.81) | 4.42 ± (4.98) | 0.833 |
| Firmicutes: Ruminococcus bromii | 3.67 ± (4.59) | 2.42 ± (3.59) | 0.105 |
| Firmicutes: Anaerostipes hadrus | 4.58 ± (4.62) | 3.79 ± (2.31) | 0.734 |
| Firmicutes: Fusicatenibacter saccharivorans | 1.37 ± (2.03) | 3.95 ± (4.13) | 0.020 |
| Firmicutes: Eubacterium rectale | 2.55 ± (2.81) | 4.77 ± (7.07) | 0.446 |
| Verrucomicrobia: Akkermansia muciniphila | 3.60 ± (6.09) | 3.90 ± (7.95) | 0.932 |
| Bacteroidetes: Alistipes putredinis | 1.23 ± (2.31) | 1.56 ± (2.92) | 0.418 |
| Firmicutes: Roseburia faecis | 1.43 ± (2.31) | 1.28 ± (3.20) | 0.529 |
| Actinobacteria: Bifidobacterium longum | 2.22 ± (2.54) | 2.65 ± (3.99) | 0.799 |
| Firmicutes: Eubacterium hallii | 2.15 ± (1.63) | 1.95 ± (1.99) | 0.944 |
| Proteobacteria: Escherichia coli | 3.97 ± (5.72) | 0.33 ± (0.38) | 0.183 |
| Actinobacteria: Bifidobacterium | 3.12 ± (3.87) | 2.64 ± (4.53) | 0.589 |
| pseudocatenulatum | | | |
| Firmicutes: Dorea longicatena | 0.71 ± (0.75) | 1.07 ± (1.28) | 0.799 |
| Firmicutes: Blautia obeum | 1.38 ± (3.51) | 0.63 ± (0.68) | 0.820 |
| Bacteroidetes: Alistipes finegoldii | 1.45 ± (2.33) | 2.90 ± (3.62) | 0.203 |
| Bacteroidetes: Prevotella copri | 0.16 ± (0.49) | 0.003 ± (0.009) | 1.000 |
| Firmicutes: Ruminococcus bicirculans | 0.28 ± (0.26) | 0.76 ± (1.35) | 0.544 |
| Bacteroidetes: Bacteroides vulgatus | 1.53 ± (2.04) | 1.62 ± (2.21) | 0.932 |
| Firmicutes: Ruminococcus torques | 0.89 ± (1.62) | 0.58 ± (0.88) | 0.799 |
| Firmicutes: Eubacterium siraeum | 2.07 ± (5.99) | 1.69 ± (4.21) | 0.589 |
| Firmicutes: Coprococcus comes | 0.88 ± (0.88) | 0.93 ± (0.95) | 0.441 |
| Firmicutes: Dialister sp. CAG. 357 | 1.43 ± (2.92) | 0.98 ± (1.68) | 0.361 |
| Bacteroidetes: Parabacteroides merdae | 0.46 ± (1.20) | 1.85 ± (4.35) | 0.201 |
| Actinobacteria: Bifidobacterium bifidum | 1.60 ± (4.27) | 1.61 ± (3.62) | 1.000 |

Shannon Index, Simpson's Index, Species Richness and Pielou's evenness were measured at the taxonomic level of species.
Dataset were rarefied to 1 million sequences per sample.
Mean RA % = average number of sequences per taxa, calculated from the total sum of all sequence counts, depicted as a percentage.
Microbial taxa (>1%) shown.
(SD) % = standard deviation as a percentage.
Wilcoxon signed-rank paired test used to compare two related samples for each PD participant's group.
Benjamini-Hochberg significance: p-value < 0.05 (bold italics).

TABLE 7
Blood makers of short chain fatty acids metabolites, gut leakiness,
systemic inflammation, brain health, with fecal intestinal inflammation,
between de novo or treated PD baseline and prebiotic samples

| | | |
|---|---|---|
| Baseline | Prebiotic | p-value |

Short Chain Fatty Acids - Plasma (ug/mL)

| | | | | | |
|---|---|---|---|---|---|
| Acetate | 1.96 | (0.77) | 2.48 | (0.54) | 0.128 |
| Propionate | 0.09 | (0.06) | 0.10 | (0.03) | 0.250 |
| Butyrate | 0.06 | (0.04) | 0.07 | (0.03) | 0.570 |
| Total SCFA | 2.12 | (0.84) | 2.62 | (0.50) | 0.128 |
| Butyrate-to-Total SCFA Ratio | 0.03 | (0.02) | 0.03 | (0.01) | 0.426 |
| Propionate + Butyrate-to-Total SCFA Ratio | 0.12 | (0.07) | 0.13 | (0.04) | 0.425 |

Intestinal Barrier Integrity & Bacterial Translocation- Plasma (ng/ml)

| | | | | | |
|---|---|---|---|---|---|
| Zonulin | 21.37 | (7.66) | 14.70 | (5.58) | 0.088 |
| LBP | 14,321 | (6,131) | 16,946 | (8,894) | 0.094 |

Intestinal Inflammation - Feces (ug/g)

| | | | | | |
|---|---|---|---|---|---|
| Calprotectin | 120.8 | (140.7) | 90.35 | (113.7) | 0.067 |

Systemic Inflammation - Serum (pg/ml)

| | | | | | |
|---|---|---|---|---|---|
| IFN-γ | 3.15 | (1.57) | 3.30 | (2.11) | 0.556 |
| IL-6 | 0.617 | (0.30) | 0.75 | (0.57) | 0.695 |
| IL-8 | 10.43 | (2.92) | 10.20 | (3.53) | 0.764 |
| IL-10 | 0.23 | (0.10) | 0.26 | (0.10) | 0.084 |
| TNF-α | 0.64 | (0.14) | 0.67 | (0.21) | 0.344 |

Brain Health - Serum (pg/ml) or (ng/ml)

| | | | | | |
|---|---|---|---|---|---|
| BDNF | 6,007 | (2,116) | 5,765 | (2,065) | 0.750 |
| NfL | 56.07 | (24.99) | 48.92 | (23.62) | 0.008 |
| HMGB-1 | 240.60 | (94.73) | 200.60 | (70.40) | 0.299 |

Short Chain Fatty Acids - Plasma (ug/mL)

| | | | | | |
|---|---|---|---|---|---|
| Acetate | 1.84 | (0.64) | 3.00 | (1.74) | 0.027 |
| Propionate | 0.07 | (0.02) | 0.13 | (0.06) | 0.011 |
| Butyrate | 0.05 | (0.03) | 0.08 | (0.03) | 0.019 |
| Total SCFA | 1.97 | (0.66) | 3.22 | (1.78) | 0.019 |
| Butyrate-to-Total SCFA Ratio | 0.03 | (0.02) | 0.03 | (0.02) | 1.000 |
| Propionate + Butyrate-to-Total SCFA Ratio | 0.10 | (0.03) | 0.15 | (0.06) | 0.054 |

Intestinal Barrier Integrity - Plasma (ng/ml)

| | | | | | |
|---|---|---|---|---|---|
| Zonulin | 20.79 | (5.80) | 13.24 | (4.26) | <0.001 |
| LBP | 14,631 | (5,996) | 13,464 | (2,594) | 0.845 |

Intestinal Inflammation - Feces (ug/g)

| | | | | | |
|---|---|---|---|---|---|
| Calprotectin | 28.07 | (22.61) | 19.56 | (11.01) | 0.169 |

Systemic Inflammation - Serum (pg/ml)

| | | | | | |
|---|---|---|---|---|---|
| IFN-γ | 9.61 | (19.73) | 3.42 | (2.82) | 0.275 |
| IL-6 | 0.71 | (0.39) | 0.64 | (0.34) | 0.322 |
| IL-8 | 43.02 | (106.9) | 9.84 | (2.81) | 0.921 |
| IL-10 | 0.33 | (0.28) | 0.40 | (0.45) | 0.921 |
| TNF-α | 0.71 | (0.24) | 0.69 | (0.24) | 0.757 |

Brain Health - Serum (pg/ml) or (ng/ml)

| | | | | | |
|---|---|---|---|---|---|
| BDNF | 5,786 | (1,531) | 5,158 | (1,866) | 0.404 |
| NfL | 82.58 | (39.22) | 75.94 | (36.66) | 0.091 |
| HMGB-1 | 206.30 | (120.30) | 218.10 | (95.59) | 0.603 |

All data are shown as mean (standard deviation).
Wilcoxon signed-rank paired test used to compare two related samples for each PD participant.
Significance: p-value < 0.05 (bold italics).
sd, standard deviation; LBP, lipopolysaccharide binding protein; IFN-γ, interferon-gamma; IL, interleukin; TNF-α, tumor necrosis factor-alpha; BDNF, brain derived-neurotrophic-factor; NfL, neurofilament light chain; HMGB-1, high mobility group box 1 protein.

Overall, the prebiotic intervention reduced the relative abundance of putative pro-inflammatory bacteria and increased the abundance of putative SCFA-producing bacteria in the feces with a concurrent increase in plasma SCFA.

c. Prebiotic Fiber Induced Changes in Barrier Integrity and Inflammation.

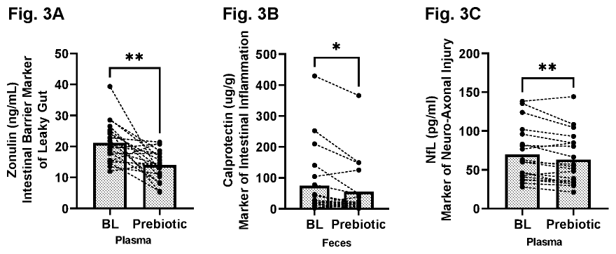

No prebiotic induced effects were noted for plasma LBP (marker for bacterial translocation, Table 5). However, plasma zonulin was significantly decreased (p<0.001) when comparing BL to post treatment zonulin levels across all PD participants, indicating that the prebiotic intervention significantly improved intestinal barrier integrity (FIG. 3A). This appears to be largely driven by treated PD participants, although there is a trend for a decrease in de novo PD participants as well (Table 7). These data are congruent with the finding that intestinal inflammation (i.e., fecal calprotectin) was significantly reduced after consumption of the prebiotic fiber mixture (p<0.044, FIG. 3B). Despite changes in the microbiota, barrier integrity (i.e., zonulin), and intestinal inflammation, there were no significant prebiotic treatment-induced changes in serum cytokine levels (IL-6, IL-8, IL-10, IFN-γ, TNF-α) (Table 5). This could be due to the short follow up time (10 days) and/or the small sample size (not powered to detect clinical changes).

d. Effects of Prebiotic Fiber Mixture on Neuroinflammation and Brain Health.

It is well established that the intestinal milieu can influence the brain, including neuro-inflammation, levels of trophic factors, and neurodegeneration. No prebiotic intervention-induced changes in the selected marker of neuroinflammation (HMGB-1), nor for the neurotrophic factor (BDNF) (Table 5). However, the selected marker of degeneration—neurofilament light chain (NfL) (Gaetani et al., J Neurol Neurosurg Psychiatry 90: 870-881 (2019))—was significantly reduced (NfL, p=0.003) after the prebiotic intervention (FIG. 3C, Table 5). The change in NL was driven by de novo PD participants (NfL, p<0.008), although treated PD participants also had a non-significant reduction (Table 7).

e. Correlations of Bacterial Taxa, Experimental Measures, and Clinical Characteristics

Correlation analysis was conducted to assess the relationship between the relative abundances of species taxa, experimental outcomes (i.e., blood markers of intestinal barrier integrity, intestinal, systemic, and neuro-inflammation, brain health, and SCFA) and demographic and clinical parameters (i.e., age, Bristol stool score, LEDD, PD duration, UPDRS).

The analyses revealed a relationship between the intestinal barrier, the intestinal microbiota, and inflammation (Table 8). Zonulin positively correlated with the putative pro-inflammatory bacterial species Escherichia coli and negatively correlated with putative SCFA-producing bacterial species Parabacteroides merdae, which are consistent with pro-inflammatory bacteria promoting intestinal barrier dysfunction and SCFA-producing bacteria reducing intestinal barrier dysfunction. SCFA-producing bacteria are purported to be anti-inflammatory. Indeed, systemic inflammation (i.e., TNF-α) negatively correlated with the putative SCFA-producing bacteria, Parabacteroides merdae. Constipation (i.e., Bristol stool score) positively correlated with LBP, a marker of bacterial translocation.

TABLE 8
Significant associations following the consumption
of prebiotics in all PD participants

| Variable 1 | Variable 2 | R Value | p value |
|---|---|---|---|
| Zonulin | Escherichia coli | 0.790 | 0.005 |
| | Parabacteroides merdae | −0.660 | 0.043 |
| Neurofilament light chain | Bristol Stool Score | 0.606 | 0.005 |
| (NfL) | | | |
| Tumor necrosis factor-alpha | Parabacteroides merdae | −0.697 | 0.030 |
| (TNF-α) | | | |
| Lipopolysaccharide binding | Bristol Stool Score | 0.448 | 0.047 |
| protein (LBP) | | | |
| Age of PD Subjects | Eubacterium siraeum | −0.874 | 0.007 |
| | Ruminococcus | −0.768 | 0.003 |
| | bircirculans | | |
| Bristol Stool Score | NfL | 0.606 | 0.005 |
| | LBP | 0.447 | 0.047 |
| Levodopa daily dosages | PD Symptom Duration | 0.690 | <0.001 |
| (LEDD) | Bifidobacterium | −0.618 | 0.047 |
| | adolescentis | | |

R = Spearman's rank correlation coefficient is a nonparametric measure of the strength and direction of association that exists between two variables, ranging from values +1 to −1.
Significance: p-values < 0.05 (bold italics).
Data used for correlation analysis was transformed using Log2 fold change from all PD participant's baseline samples (n = 20).

Constipation is associated with cognitive dysfunction. Indeed, in this cohort, constipation (e.g., Bristol stool score) positively correlated with NfL, a marker of neurodegeneration. Age-associated changes in the microbiome are reported in the literature and in this study, age was negatively associated with both putative SCFA-producing bacterial species Eubacterium siraeum and Ruminococcus bircirculans (which could contribute to inflammaging). Finally, a total of 10 out of 20 PD subjects were taking levodopa (i.e., treated PD participants). Levodopa daily dosage was negatively correlated with the relative abundance of the putative SCFA-producing bacterial species Bifidobacterium adolescentis.

Thus, after 10 days of daily consumption of a prebiotic fiber mixture, GI and PD motor symptoms improved, putative pro-inflammatory bacteria were decreased in feces (Proteobacteria and Escherichia coli), while relative abundance short-chain-fatty-acids (SCFA)-producing bacteria were increased, including species Faecalibacterium prausnitizii. Blood sample assessments showed increased total SCFA metabolites, improved intestinal barrier integrity, reduced levels of a peripheral marker of neurodegeneration, and reduced intestinal inflammation. Prebiotics safely altered the microbiome in patients with PD.

The prebiotic fiber mixture designed to increase SCFAs was well tolerated and improved GI symptoms in a subset of levodopa-treated PD patients. Ten days of the prebiotic intervention improved the UPDRS score. This clinical improvement was associated with positive changes in the intestinal microbiota community, improved intestinal barrier integrity, reduced intestinal inflammation, and reduced systemic marker of neurodegeneration. Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein.

The term “about” as used herein can allow for a degree of variability in a value or range, for example, within 10%, within 5%, or within 1% of a stated value or of a stated limit of a range.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation.

Any use of section headings and subheadings is solely for ease of reference and is not intended to limit any disclosure made in one section to that section only; rather, any disclosure made under one section heading or subheading is intended to constitute a disclosure under each and every other section heading or subheading.

Various modifications and variations of the described compositions, methods, and uses of the technology will be apparent to those skilled in the art without departing from the scope and spirit of the technology as described. Although the technology has been described in connection with specific exemplary embodiments, the invention as claimed should not be unduly limited to such specific embodiments. Indeed, various modifications of the described modes for carrying out the invention that are obvious to those skilled in the art are intended to be within the scope of the following claims.

The terms and expressions, which have been employed, are used as terms of description and not of limitation. In this regard, where certain terms are defined and are described or discussed elsewhere, the definitions and all descriptions and discussions are intended to be attributed to such terms. There also is no intention in the use of such terms and expressions of excluding any equivalents of the features shown and described or portions thereof.

Further, all publications and patents mentioned herein are incorporated by reference in their entireties for all purposes. In the event of inconsistent usages between this document and those documents so incorporated by reference, the usage in the incorporated reference should be considered supplementary to that of this document; for irreconcilable inconsistencies, the usage in this document controls.

What is claimed is:

1. A composition comprising (i) a resistant starch, (ii) a resistant non-starch α-linked glucan, (iii) a cereal bran, which is optionally stabilized, and (iv) inulin, a fructo-oligosaccharide, or both.

2. The composition of claim 1, wherein the resistant starch is type 1, type 2, type 3, type 4, type 5, or any combination thereof.

3. The composition of claim 2, wherein the resistant starch is type 2.

4. The composition of claim 3, wherein the type 2 resistant starch is raw potato starch.

5. The composition of claim 1, wherein the resistant non-starch α-linked glucan is resistant dextrin/maltodextrin.

6. The composition of claim 1, wherein the cereal bran is rice bran, wheat bran, corn bran, oat bran, barley bran, sorghum bran, millet bran, rye bran, triticale bran, or any combination thereof.

7. The composition of claim 6, wherein the cereal bran is rice bran, which is optionally stabilized.

8. The composition of claim 1, wherein the inulin is agave branched inulin.

9. The composition of claim 1, comprising raw potato starch, resistant dextrin/maltodextrin, rice bran, which is optionally stabilized, and agave branched inulin.

10. The composition of claim 1, 2, 3, 4, 5, 6, 7, 8, or 9, wherein each of (i)-(iv) is present in an amount in the range of about 5% to about 70% of the total amount by weight.

11. The composition of claim 3 or 9, which comprises about 30% type 2 resistant starch, about 30% resistant maltodextrin, about 30% rice bran, which is optionally stabilized, and about 10% agave branched inulin.

12. An ingestible formulation comprising the composition of claim 10.

13. The ingestible formulation of claim 12, which comprises from about 2 grams to about 20 grams of the composition.

14. The ingestible formulation of claim 12, wherein the ingestible formulation is a supplement, a powder sachet, a powder for a shake, a liquid shake, a prebiotic shot, a snack, or a meal replacement.

15. The ingestible formulation of claim 14, which comprises from about 2 grams to about 20 grams of the composition.

16. An ingestible formulation comprising the composition of claim 11.

17. The ingestible formulation of claim 16, which comprises from about 2 grams to about 20 grams of the composition.

18. The ingestible formulation of claim 16, wherein the ingestible formulation is a supplement, a powder sachet, a powder for a shake, a liquid shake, a prebiotic shot, a snack, or a meal replacement.

19. The ingestible formulation of claim 18, which comprises from about 2 grams to about 20 grams of the composition.

20. A method of improving gastrointestinal health in a human with a condition, disease, or disorder, which method comprises administering to the human a composition of claim 1, 2, 3, 4, 5, 6, 7, 8 or 9 or an ingestible formulation comprising same.

21. The method of claim 20, wherein the ingestible formulation comprises from about 2 grams to about 20 grams of the composition.

22. The method of claim 20, wherein the ingestible formulation is a supplement, a powder sachet, a powder for a shake, a liquid shake, a prebiotic shot, a snack, or a meal replacement.

23. The method of claim 22, wherein the ingestible formulation comprises from about 2 grams to about 20 grams of the composition.

24. The method of claim 20, wherein the ingestible formulation is administered at least once daily.

25. The method of claim 24, wherein the ingestible formulation is administered twice daily.

26. The method of claim 20, wherein the human has inflammatory bowel disease, irritable bowel syndrome, liver disease, a metabolic disorder, a cardiovascular disease, a cancer, a neurodegenerative disease, an infection, a condition induced by exposure to chemotherapy or radiation, or an allergy.

27. The method of claim 26, wherein the inflammatory bowel disease is ulcerative colitis, Crohn disease, or pouchitis.

28. The method of claim 26, wherein the liver disease is alcoholic liver disease or non-alcoholic steatohepatitis (NASH).

29. The method of claim 26, wherein the metabolic disorder is obesity, metabolic syndrome, or diabetes.

30. The method of claim 26, wherein the neurodegenerative disease is Parkinson disease, Alzheimer disease, ataxia, Huntington disease, motor neuron disease, multiple system atrophy, a neuromuscular disorder, Parkinsonism, post-traumatic stress disorder (PTSD), progressive supranuclear palsy, or spasticity.

31. The method of claim 26, wherein the infection is a viral infection.

32. The method of claim 31, wherein the viral infection is human immunodeficiency virus infection.

33. The method of claim 26, wherein the condition induced by exposure to chemotherapy or radiation is enteritis.

34. The method of claim 20, wherein each of (i)-(iv) in the composition is present in an amount in the range of about 5% to about 70% of the total amount by weight.

35. The method of claim 20, wherein the composition comprises about 30% type 2 resistant starch, about 30% resistant maltodextrin, about 30% rice bran, which is optionally stabilized, and about 10% agave branched inulin.
